SEQUENCE LISTING (4 sequences)

SEQ ID NO: 1 - protein, 438 aa, Homo sapiens
  REGION 1..438: note = MISC_FEATURE - CLN3
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 2 - DNA, 1317 bp, Homo sapiens
  misc_feature 1..1317: note = CNL3
  source 1..1317: mol_type = other DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 3 - DNA, 546 bp, synthetic construct
  misc_feature 1..546: note = Synthetic Polynucleotide
  misc_feature 1..546: note = P546 promoter
  source 1..546: mol_type = other DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 4 - DNA, 2479 bp, synthetic construct
  misc_feature 1..2479: note = Synthetic Polynucleotide
  misc_feature 1..2479: note = CLN3 gene cassette
  source 1..2479: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

(19) United States
(12) Patent Application Publication
(10) Pub. No.: US 2024/0424137 A1
(43) Pub. Date: Dec. 26, 2024

(54) Biomarkers For Lysosomal Storage Diseases

(71) Applicants: Amicus Therapeutics, Inc., Philadelphia, PA (US); Sanford Health, Sioux Falls, SD (US)

(72) Inventors: Jon Brudvig, Philadelphia, PA (US); Jill Weiner, Philadelphia, PA (US); Tyler Johnson, Philadelphia, PA (US); Jacob Cain, Philadelphia, PA (US); Derek Timm, Philadelphia, PA (US)

(73) Assignees: Amicus Therapeutics, Inc., Philadelphia, PA (US); Sanford Health, Sioux Falls, SD (US)

(21) Appl. No.: 18/698,976

(22) PCT Filed: Oct. 7, 2022
(86) PCT No.: PCT/US22/77760

§ 371 Date: Apr. 5, 2024

Related U.S. Application Data
(60) Provisional application No. 63/253,749, filed on Oct. 8, 2021.

(51) Int. Cl. A61K 48/00; A61B 5/00; A61B 5/398; A61K 9/00; C12N 15/86; G01N 33/68; G01N 33/92

(52) U.S. Cl. CPC A61K 48/005; A61B 5/398; A61B 5/4064; A61K 9/0085; A61K 48/0075; C12N 15/86; G01N 33/6893; G01N 33/92; C12N 2750/14143; G01N 2333/9015; G01N 2333/916; G01N 2333/96416; G01N 2333/988; G01N 2800/04

(57) ABSTRACT

Methods of determining a disease score of a patient diagnosed with lysosomal storage disease (LSD) are described. The disease score includes one or more of the following: (i) one or more biofluid biomarkers; (ii) one or more neurophysiological measurements; and (iii) one or more neurobehavior measurement. Also described method of treating LSD including determining the disease score and administering to the patient a therapy. The therapy includes one or more of the following: (i) enzyme replacement therapy, (ii) gene therapy; and (iii) a small molecule.

### INCORPORATION BY REFERENCE OF THE SEQUENCE LISTING

This application contains, as a separate part of disclosure, a Sequence Listing in computer-readable form (filename: AT21-007_Sequence Listing) which is incorporated by reference herein in its entirety.

### FIELD

The present disclosure relates to diagnosis and tracking disease progression state of lysosomal storage diseases using biomarkers.

### BACKGROUND

Many human diseases result from mutations that cause changes in the amino acid sequence of a protein which reduce its stability and may prevent it from folding properly. Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded proteins are often eliminated by the quality control mechanisms after initially being retained in the ER. In certain instances, misfolded proteins can accumulate in the ER before being eliminated. The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Such mutations can lead to lysosomal storage diseases (LSDs), which are characterized by deficiencies of lysosomal enzymes due to mutations in the genes encoding the lysosomal enzymes. The resultant disease causes the pathologic accumulation of substrates of those enzymes, which include lipids, carbohydrates, and polysaccharides. Although there are many different mutant genotypes associated with each LSD, many of the mutations are missense mutations which can lead to the production of a less stable enzyme. These less stable enzymes are sometimes prematurely degraded by the ER-associated degradation pathway. This results in the enzyme deficiency in the lysosome, and the pathologic accumulation of substrate. Such mutant enzymes are sometimes referred to in the pertinent art as “folding mutants” or “conformational mutants.”

Neuronal ceroid lipofuscinoses (NCLs) are a group of severe neurodegenerative disorders caused by mutations in one of at least 13 genes. Mutations in the CLN3 gene cause juvenile NCL or CLN3-Batten Disease (Kitzmü et al., Human Molecular Genetics 2008; 17(2):303-312; Munroe et al., Am J Hum Genet. 1997; 61:310-316), which has also been called Spielmeyer-Sjogren-Vogt disease. Mutations disturb a range of cellular processes including lysosomal function. At this time, 67 disease causing mutations have been described. However, 85% of patients are homozygous for the 1.02 kb deletion leading to the loss of exon 7 and exon 8. CLN3 mutations found in patients predominantly cause reduced abundance or functionality of the CLN3 protein.

The typical age of onset in CLN3-Batten disease is between 4-7 years with insidious, but rapidly progressive vision loss. Children with juvenile NCL can go from having normal vision to blindness in a matter of months, but can also maintain light-dark perception for several years after. Cognitive and motor decline usually follows next (7-10 years of age) alongside with behavioral problems such as aggression (8-10 years of age), and then seizures (10-12 years of age). Parkinsonian features develop between 11-13 years of age. Cardiac conduction abnormalities have been reported in individuals at later stages of the disease. There is a high phenotypic variability in individuals affected with CLN3-Batten disease, but all have vision problems. Moreover, the physical subscale of the Unified Batten Disease Rating Scale (UBDRS) that has been validated in 82 patients, shows a steady and measurable decline of 2.86 points per year (2.27-3.45, p<0.0001). The average survival is usually 15 years from symptom onset to end of life.

Therapeutic measures for CLN3-Batten disease have been wide-ranging in an effort to ameliorate disease. These include drug therapy such as EGIS-8332 and talampanel which target AMPA receptors, drugs that allow read-through of premature stop mutations, drugs to assist in break-down of accumulated storage material (cystagon/cysteamine), and even immune suppression therapy (mycophenolate, prednisolone). Enzyme replacement and stem cell therapies have also been evaluated. While many therapeutic approaches have been studied, few have been evaluated in a clinical setting. None are available that slow progression or cure the disease. Patients and families rely on treatments to ameliorate symptoms and palliative care.

The Cln3^{Δex7/8} mouse model was created in the early 2000s to mimic the most common disease-causing mutation in CLN3-Batten disease patients: an approximately 1 kb mutation that eliminates exons 7 and 8 from the CLN3 gene (Cotman et al., Hum Mol Genet. 2002; 11(22):2709-2721; Mole et al., Eur J Paediatr Neurol. 2001; 5:7-10). The mutation is found in a homozygous manner in 85% of the patients and as a heterozygous mutation in combination with point mutations on the other allele in an additional 15% of patients. The exon loss is predicted to produce a frameshift mutation, leading to a truncated protein product with lost or reduced activity (Lerner et al., Cell. 1995 Sep. 22; 82(6):949-57; Kitzmüller et al., Hum Mol Genet. 2008 Jan. 15; 17(2):303-12). In their original study, Cotman et al. demonstrated the CLN3^{Δex7/8} mouse model successfully recapitulated several aspects of CLN3 disease. CLN3^{Δex7/8} animals accumulated autofluorescent storage material and ATP Synthase subunit C in the nervous system at various time points, and exhibited astrocyte reactivity in the brain starting at 10 months of age. Subsequent studies detailed altered glutamate receptor function in the cerebellum, corresponding with motor deficits on an accelerating rotarod assay (Cotman et al., Hum Mol Genet. 2002; 11(22):2709-2721). Behaviorally, Cln3^{Δex7/8} mice have been characterized at both young and mature time points, where neurodevelopmental motor delays were seen in neonatal and young adult mice, and deficits in gait and hind limb clasping were seen at 10-12 months of age (Cotman et al., Hum Mol Genet. 2002; 11(22):2709-2721; Osório et al., Genes Brain Behav. 2009 April; 8(3): 337-345). CLN3^{Δex7/8} mice have variable vision phenotypes depending on the colony and lab; likewise, survival deficits have been noted in some locations (Cotman et al., Hum Mol Genet. 2002; 11(22):2709-2721; Seigel et al., Mol Cell Neurosci. 2002 April; 19(4):515-27). Taken together, the Cln3^{Δex7/8} mouse model carrying the most frequent human mutation, exhibits numerous cellular and behavioral changes consistent with CLN3-Batten disease, making it a suitable model for testing therapies.

However, neurobehavior phenotypes may not always be consistent with the actual state of disease progression, typically manifest late, and are highly variable between labs Thus, there remains a need for sensitive means of tracking disease progression, determining response to a treatment, and methods of treating the lysosomal storage disease.

### SUMMARY

Provided herein the method of determining a disease score of a patient diagnosed with lysosomal storage disease (LSD). In some embodiments, the disease score comprises one or more of the following: (i) one or more biofluid biomarkers; (ii) one or more neurophysiological measurements; and (iii) one or more neurobehavior measurement.

In some embodiments, the one or more biomarkers is selected from the group consisting of one or more metabolites, one or more proteins, one or more lipids and one or more lipid conjugates. In some embodiments, the one or more metabolites is selected from the group consisting of glycerophosphoinositol, glycerophosphocholine (GPC), glycerophosphoserine, and glycerophosphoethanolamine (GPE). In some embodiments, the one or more proteins is selected from the group consisting of neurofilament light (NFL), ubiquitin c-terminal hydrolase L1 (UCHL1), mitochondrial ATP synthase subunit C (SCMAS), gamma enolase (ENO2), cathepsin D (CTSD), Progranulin (GRN), palmitoyl-protein thioesterase 1 (PPT1), tripeptidyl-peptidase 1 (TPP1), troponin T, and troponin I. The one or more lipids is selected from the group Phosphatidylcholine, Phosphatidylcholine, Phosphatidylcholine and PE Phosphatidylethanolamine. In some embodiments, the one or more lipid conjugates is selected from the group consisting of 1-stearoyl-2-docosahexaenoyl-GPC (18:0/22:6), 1-palmitoyl-2-docosahexaenoyl-GPE (16:0/22:6), 1-stearoyl-2-docosahexaenoyl-GPC (18:0/22:6) and 1-oleoyl-2-docosahexaenoyl-GPC (18:1/22:6).

In some embodiments, the one or more neurophysiological measurements is selected from the group consisting of gait analysis, neuro-imagining metrics, retinal function, peripheral nerve function, clinical assessment of neurological function, and patient-reported episodes. In some embodiments, the neuro-imagining metrics include one or more of: brain ventricle size, grey matter hyperintensities or hypointensities, white matter hyperintensities or hypointensities, periventricular hyperintensities, cerebellar atrophy, cortical atrophy, whole brain volume, corpus callosum volume, white matter integrity, radial diffusivity, axial diffusivity, and magnetic resonance spectroscopy measurements. In some embodiments, the retinal function includes one or more of: ERG waveform, visual-evoked potential measurement, and optical coherence tomography measurement.

In some embodiments, the one or more neurobehavioral measurements include measuring one or more of: motor function, language function, cognitive function, clinical rating scale, and PROM.

In some embodiments, the LSD is CLN1, CLN2, CLN3, CLN4, CLN5, CLN6, CLN7, CLN8, CLN10, CLN11, CLN12, CLN13, and/or CLN14; Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy, Farber lipogranulomatosis, Wolman and cholesteryl ester storage disease, pycnodystostosis, cystinosis, Salla disease, Danon disease, Griscelli disease Types 1/2/3, and Hermansky Pudliak Disease Type 2.

Another aspect of the present disclosure relates to method of treating lysosomal storage disease (LSD) patient. In some embodiments, the method comprises determining the disease score, and administering the patient a therapy.

In some embodiments, the therapy comprises one or more of the following: (i) enzyme replacement therapy, (ii) gene therapy; and (iii) a small molecule. In some embodiments, the gene therapy is delivered systemically or to central nervous system. In some embodiments, the gene therapy is delivered to a brain. In some embodiments, the therapy is delivered to a spinal cord. In some embodiments, the gene therapy is delivered intrathecally. In some embodiments, the gene therapy includes a composition comprising rAAV9. In some embodiments, the rAAV9 comprises a self-complementary genome comprising said polynucleotide.

### BRIEF DESCRIPTION OF THE DRAWINGS

FIG. 1A-1B provides longitudinal analysis of CLN3 patients according to one or more embodiments of the present disclosure;

FIG. 2 provides metabolomics profiling of CLN3 pigs according to one or more embodiments of the present disclosure;

FIG. 3 provides metabolomics profiling of CLN3 pigs according to one or more embodiments of the present disclosure;

FIG. 4 provides metabolomics profiling of CLN3 pigs according to one or more embodiments of the present disclosure;

FIG. 5 shows representative images of immune-stained brain sections from WT and Cln3^{Δex7-8/Δex7-8} pigs to determine early and/or persistent SubC accumulation according to one or more embodiments of the present disclosure;

FIG. 6 shows immunoreactivity quantification of immune-stained brain sections from WT and Cln3^{Δex7-8/Δex7-8} pigs to determine early and/or persistent SubC accumulation according to one or more embodiments of the present disclosure;

FIG. 7 shows representative images of immune-stained brain sections from WT and Cln3^{Δex7-8/Δex7-8} pigs to determine astrocyte reactivity according to one or more embodiments of the present disclosure;

FIG. 8 shows immunoreactivity quantification of immune-stained brain sections from WT and Cln3^{Δex7-8/Δex7-8} pigs to determine astrocyte reactivity according to one or more embodiments of the present disclosure;

FIG. 9 shows representative images brain sections from WT and Cln3^{Δex7-8/Δex7-8} pigs to determine cortical plate thickness according to one or more embodiments of the present disclosure;

FIG. 10 shows cortical plate thickness from WT and Cln3^{Δex7-8/Δex7-8} pigs according to one or more embodiments of the present disclosure;

FIG. 11 shows representative images of immune-stained brain sections from WT and Cln3^{Δex7-8/Δex7-8} pigs to determine cortical neurodegeneration according to one or more embodiments of the present disclosure;

FIG. 12 shows calbindin cell counts in immune-stained brain sections from WT and Cln3^{Δex7-8/Δex7-8} pigs to determine cortical neurodegeneration according to one or more embodiments of the present disclosure;

FIG. 13 shows electroretinogram (ERG) for WT and Cln3^{Δex7-8/Δex7-8} pigs according to one or more embodiments of the present disclosure;

FIG. 14 shows images of retina sections for WT and Cln3^{Δex7-8/Δex7-8} pigs according to one or more embodiments of the present disclosure;

FIG. 15 shows outer nuclear layer width and inner nuclear layer width in WT and Cln3^{Δex7-8/Δex7-8} pigs according to one or more embodiments of the present disclosure;

FIG. 16 shows principal component analysis of gait measurements according to one or more embodiments of the present disclosure;

FIG. 17 provides summary of principal component analysis for CLN6 and CLN8 patients according to one or more embodiments of the present disclosure;

FIG. 18 provides proteomic profiling of CLN1 mice according to one or more embodiments of the present disclosure;

FIG. 19 provides proteomic profiling of CLN1 mice according to one or more embodiments of the present disclosure;

FIG. 20 provides proteomic profiling of CLN1 mice according to one or more embodiments of the present disclosure;

FIG. 21 provides proteomic profiling of CLN1 mice according to one or more embodiments of the present disclosure;

FIG. 22 provides proteomic profiling of CLN1 mice according to one or more embodiments of the present disclosure;

FIG. 23 provides proteomic profiling of CLN6 mice according to one or more embodiments of the present disclosure;

FIG. 24 provides proteomic profiling of CLN6 mice according to one or more embodiments of the present disclosure;

FIG. 25 provides proteomic profiling of CLN6 mice according to one or more embodiments of the present disclosure;

FIG. 26 provides proteomic profiling of CLN6 mice according to one or more embodiments of the present disclosure;

FIG. 27 provides proteomic profiling of CLN6 mice according to one or more embodiments of the present disclosure;

FIG. 28 provides evaluation of glycerophosphodiester species in in CLN3^{Δex7-8} pigs according to one or more embodiments of the present disclosure;

FIG. 29 provides evaluation of glycerophosphodiester species in in CLN3^{Δex7-8} pigs according to one or more embodiments of the present disclosure;

FIG. 30 provides evaluation of glycerophosphodiester species in in CLN3^{Δex7-8} pigs according to one or more embodiments of the present disclosure;

FIG. 31 provides evaluation of glycerophosphodiester species in in CLN3^{Δex7-8} pigs according to one or more embodiments of the present disclosure;

FIG. 32 provides evaluation of glycerophosphodiester species in in CLN3^{Δex7-8} pigs according to one or more embodiments of the present disclosure;

FIG. 33 provides metabolomics profiling of CLN3 human patients according to one or more embodiments of the present disclosure; and

FIG. 34 provides an analysis of correlation between GPI levels and UBDRS Capability (A) or Physical (B) scores according to one or more embodiments of the present disclosure.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

A “patient” refers to a subject who has been diagnosed with a lysosomal storage disease. The patient may be human or animal.

Principal component analysis (PCA) identifies correlated variables which explain the most variation in the original data set. Accordingly, in one or more embodiments, a disease score model is developed using PCA. Contrastive PCA (cPCA) is a novel statistical method that aims to discover what is in one dataset relative to another—to find which combination of variables add the most contrast between healthy and disease groups. Accordingly, in one or more embodiments, a disease score model is developed using PCA, cPCA, or a combination thereof.

### Disease Score

A “comprehensive cPCA disease score” are “disease score” are used interchangeably to refer to a disease score model developed using PCA, cPCA, or combination thereof. Some embodiments of the disclosure relate to methods of determining a disease score for the patient. The “disease score” for lysosomal storage disease comprises one or more biofluid biomarkers, one or more neurophysiological measurement, one or more neurobehavior measurements, or combinations thereof. In some embodiments, the disease score further comprises a symptom that is associated with lysosomal storage disease (but is not associated with a healthy individual). In some embodiments, the disease score provides a reliable indicator of lysosomal storage disease either alone or in combination with other abnormal markers or symptoms of lysosomal storage disease.

In some embodiments, the lysosomal storage disease is associated with lysosomal dysfunction, synaptic dysfunction, synaptic degeneration, microglial activation, astocytosis, neurodegeration, or combinations thereof. Accordingly, in some embodiments, the patient experiences one or more of the abnormal presence of, increased levels of, abnormal absence of, or decreased levels of one or more biofluid biomarker. In some embodiments, the one or more biomarkers are reliable indicators of lysosomal dysfunction, synaptic dysfunction, synaptic degeneration, microglial activation, astocytosis, neurodegeration, or combinations thereof.

In one or more embodiments, the lysosomal storage disease is selected from the group consisting of Batten disease, Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy, Farber lipogranulomatosis, Wolman and cholesteryl ester storage disease, pycnodystostosis, cystinosis, Salla disease, Danon disease, Griscelli disease Types 1/2/3, and Hermansky Pudliak Disease Type 2. In one or more embodiments, the lysosomal storage disease is Batten disease. In one or more embodiments, Batter disease comprises CLN1, CLN2, CLN3, CLN4, CLN5, CLN6, CLN7, CLN8, CLN10, CLN11, CLN12, CLN13, and/or CLN14. In one or more embodiments, the lysosomal storage disease is neuronal ceroid lipofuscinosis.

### Biomarkers

In some embodiments, the one or more biofluid biomarker can be represented by a biofluid disease score. In some embodiments, the biofluid disease score is developed using PCA, cPCA, or a combination thereof. In some embodiments, longitudinal biomarker characterization is performed based on T2-MRI (T2-Weighted Magnetic Resonance Imaging), ¹H-MRS (Proton Magnetic Resonance Spectroscopy), DTI (Diffision Tensor Imaging), FDG-PET (Fluorodeoxyglucose Positron Emission Tomography), KGA (Fine Motor Kinematic Gait Analysis), or combinations thereof. In some embodiments, the biofluid biomarkers are further reduced using cPCA to give a biofluid cPCA disease score. In some embodiments, two-way mixed ANOVA is performed to give a final biofluid cPCA disease score. In some embodiments, the final biofluid cPCA disease score is relative to healthy individual. Accordingly, in some embodiments, the final biofluid cPCA score can be further validated by determining absolute quantitation.

In some embodiments, the biomarker of lysosomal storage disease comprises lysosomal proteins, neurotransmitters and related metabolites, synaptic proteins, inflammatory cytokines, chemokines, glial factors, and neuron-specific proteins. In some embodiments, the biomarker for lysosomal dysfunction comprises lysosomal proteins. In some embodiments, the biomarker for synaptic dysfunction/degeneration comprises neurotransmitters and related metabolites. In some embodiments, the biomarker for synaptic dysfunction/degeneration comprises synaptic proteins. In some embodiments, the biomarker for microglial activation comprises one or more of inflammatory cytokines, chemokines, and glial factors. In some embodiments, the biomarker for astrocytosis comprises one or more of inflammatory cytokines, chemokines, and glial factors. In some embodiments, the biomarker for neurodegeneration comprises neuron-specific proteins.

In some embodiments, the biomarker comprises one or more proteins. In some embodiments, the one or more proteins comprise neurofilament light (NFL), ubiquitin c-terminal hydrolase L1 (UCHL1), mitochondrial ATP synthase subunit C (SCMAS), gamma enolase (ENO2), cathepsin D (CTSD), Progranulin (GRN), palmitoyl-protein thioesterase 1 (PPT1), tripeptidyl-peptidase 1 (TPP1), troponin T, troponin I, or combinations thereof.

In some embodiments, the biomarker comprises palmitoyl protein thioesterase 1 (PPT1) (CLN1), tripeptidyl peptidase 1 (TPP1) (CLN2), Cathepsin D (CTSD) (CLN10), progranulin (PGRN) (CLN11) and cathepsin F (CTSF) (CLN13), alpha-galactosidase A, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, or an enzymatically active fragment thereof. In one or more embodiments, the biomarker comprises a Batten-related protein selected from PPT1, TPP1, CTSD, PGRN or CTSF.

In some embodiments, the biomarker comprises a Batten-related protein. In one or more embodiments, the biomarker comprises a ceroid-lipofuscinosis neuronal protein. In one or more embodiments, the ceroid-lipofuscinosis neuronal protein is ceroid-lipofuscinosis neuronal protein 1, ceroid-lipofuscinosis neuronal protein 2, ceroid-lipofuscinosis neuronal protein 3, ceroid-lipofuscinosis neuronal protein 4, ceroid-lipofuscinosis neuronal protein 5, ceroid-lipofuscinosis neuronal protein 6, ceroid-lipofuscinosis neuronal protein 7, ceroid-lipofuscinosis neuronal protein 8, ceroid-lipofuscinosis neuronal protein 9, ceroid-lipofuscinosis neuronal protein 10, ceroid-lipofuscinosis neuronal protein 11, ceroid-lipofuscinosis neuronal protein 12, ceroid-lipofuscinosis neuronal protein 13, or ceroid-lipofuscinosis neuronal protein 14.

In some embodiments, the biomarker comprises one or more metabolites. In some embodiments, the one or more metabolite biomarkers comprise one or more glycerophosphoinositol, one or more glycerophosphocholine, one or more glycerophosphoserine, one or more glycerophosphoethanolamine, or combinations thereof.

In some embodiments, the biomarker comprises one or more lipids. In some embodiments, the one or more lipids comprises Phosphatidylcholine, Phosphatidylcholine, Phosphatidylcholine, PE Phosphatidylethanolamine, or combinations thereof.

Accordingly, in some embodiments, the biomarker comprises lipid conjugated metabolites. In some embodiments, the lipid conjugated metabolite comprises docosahexaenoic acid (DHA) derivatives. In some embodiments, the lipid conjugated metabolite comprises 1-stearoyl-2-docosahexaenoyl-GPC (18:0/22:6), 1-palmitoyl-2-docosahexaenoyl-GPE (16:0/22:6), 1-stearoyl-2-docosahexaenoyl-GPC (18:0/22:6), 1-oleoyl-2-docosahexaenoyl-GPC (18: 1/22:6), or combinations thereof.

DHA is a primary structural component of the human brain, where it is an abundant phospholipid conjugate in cell membranes. When released from phospholipids via phospholipases, DHA is converted into a variety of eicosanoid signaling molecules, which are important mediators of inflammatory processes in the brain and periphery. Given the large decreases in free DHA and DHA phospholipids in patient serum, it is likely that levels of DHA-derived eicosanoids are also altered, perhaps to an even greater extent. Thus, these molecules could have great utility as biomarkers, both alone and in combination with other targets in a comprehensive disease scoring approach. Given their important roles in inflammatory processes, changes in levels of these molecules could also be important for pathogenesis and could inform the development of new treatment strategies.

In some embodiments, the biomarker comprises KITLG, GFRA1, APBB1IP, IL17F, ENO2, or combinations thereof. In some embodiments, the biomarker comprises oleoyl-arachidonoyl-glycerol (18:1/20:4), oleoyl-arachidonoy-glycerol (18:1/20:4), N,N-dimethylvaline, 5-methylcytidine, equol sulfate, 12-HHTrE, quinolinate, allantoid, 4-vinylguaiacol sulfate, glycerol, phosphoethanolamine, 3-ketospinganine, 1-myristoyl-2palmitoyl-GPC (14:0/16:0), or combinations thereof.

In some embodiments, the biomarker comprises ITGB1BP2, IL23R, CCL2, DLK1, IL17A, LPL, AXIN1, IL17F, CCL3, ENO2, or combinations thereof. In some embodiments, the biomarker comprises biliverdin, 1-linoleoyl-GPS (18:2), thyroxine, N-methyl-GABA, N6, N6, N6-trimethyllysine, or combinations thereof.

Other biomarkers may be present at the sub-cellular level (“sub-cellular surrogate markers”) and include aberrant trafficking of lysosomal protein in cells from the ER to the lysosome; aberrant trafficking of lipids though the endosomal pathway; the presence of increased amounts misfolded lysosomal protein in the ER or cytosol; the presence of cellular stress resulting from toxic accumulation of lysosomal protein; aberrant endosomal pH levels; aberrant cell morphology; suppression of the ubiquitin/proteasome pathway; or an increase in the amount of ubiquitinated proteins.

### Molecular Biology Monitoring Assays to Detect Sub-Cellular Markers

Monitoring of lysosomal storage disease treatment can be done at the subcellular level in addition to the systemic or macroscopic level, described above. For example, disturbances in endosomal-lysosomal membrane trafficking of lipids to the Golgi complex are a characteristic of lysosomal storage disease (Sillence et al., J Lipid Res. 2002; 43(11):1837-45). Accordingly, one way of monitoring treatment of lysosomal storage disease would be to contact cells from patients with labeled lipid (BODIPY-cholesterol) and monitor its trafficking in endosomal structures. Pathological accumulation in endosomal structures, for example, would be indicative that the patient is not responding well to treatment.

As one example, pH-sensitive fluorescent probes that are endocytosed by the cells can be used to measure pH ranges in the lysosomes and endosomes (i.e. fluorescein is red at pH 5, blue to green at 5.5 to 6.5). Lysosome morphology and pH will be compared in wild type and chaperone treated and untreated patient cells. This assay can be run in parallel with the plate reader assay to determine the pH-sensitivity. For example, BODIPY-LacCer is trafficked to the Golgi in normal cells, but accumulates in the lysosomes of cells with lipid storage diseases. BODIPY-LacCer fluoresces green or red depending on the concentration in the membrane, and the green/red color ratio in the lysosome can be used to measure changes in concentration. Living healthy cells and patient cells, treated and untreated with compounds, will be incubated with BODIPY-LacCer and the red/green color ratio can be measured by the FACS and/or confocal microscope and the staining pattern (lysosome vs. Golgi) can be determined using a confocal microscope.

Trafficking occurs in cells along pH gradients (i.e. ER pH about 7, Golgi pH about 6.2-7.0, trans-Golgi network pH about 6.0, early and late endosomes pH about 6.5, lysosomes pH about 4.5) and luminal and endosomal pH is disrupted in cells with trafficking defects such as Fabry cells. Accordingly, an assay to determine pH sensitivity in wild type, SPC-treated and untreated patient cells, if correlated to positive effects of pH on trafficking, can be used to monitor restoration of trafficking in Fabry patients. If patient cells are more sensitive to changes in pH, than it would be possible to create a screening assay for SPCs that reduce the cells pH sensitivity, restores lysosome morphology or function, or more generally restores normal trafficking. In addition, mitigation of the trafficking defect can be assessed at the molecular level by determining co-localization of the deficient enzyme (α-Gal A) with a lysosomal marker such as Lyso-Tracker®. Localization of α-Gal A in the lysosome is evidence that trafficking from the ER to the lysosome is restored by treatment with the specific pharmacological chaperone. In brief, normal and patient cells, treated and untreated with SPCs, are fixed and stained with primary antibodies to the enzyme and endosome/lysosome markers (e.g., Rab7, Rab9, LAMP-1, LAMP-2, dystrophin-associated protein PAD) and fluorescently tagged secondary antibodies. The FACS and/or confocal microscope is used to quantify the amount of fluorescence due to the concentration of enzyme and other endocytic pathway markers, and the confocal microscope can be used to determine changes in staining patterns. In addition, traditional biochemical methods, such as pulse-chase metabolic labeling combined with Endoglycosidase H treatment. Endo H only cleaves proteins which have acquired ER glycosylation (high mannose N-linked), i.e., which are localized to the ER, but will not cleave proteins that have made it out of the ER to the Golgi and have acquired additional glycosylation in the Golgi. Accordingly, the greater the level of Endo H sensitive α-Gal A, the more accumulation of the protein in the ER. If the α-Gal A has made it into the Golgi, the glycosidase PNGase F can be used to. confirm whether the protein has exited the Golgi since it cleaves all N-linked sugars.

ER Stress. The toxic accumulation of misfolded proteins in the ER cells, such as the misfolded α-Gal A in Fabry patients, often results in ER stress. This leads to induction of the cell stress response which attempts to resolve the disruption in cell homeostasis. Accordingly, measuring markers of ER stress in patients following treatment would provide another way to monitor the effects of treatment. Such markers include genes and proteins associated with the Unfolded Protein Response, which include BiP, IRE1, PERK/ATF4, ATF6, XBP1 (X-box binding factor 1) and JNK (c-Jun N-terminal kinase). One method to assess ER stress is to compare expression levels between wild type and cells from a patient diagnosed with lysosomal storage disease, and also between treated and untreated cells. ER stress inducers (e.g., tunicamycin for the inhibition of N-glycosylation and accumulation of unfolded proteins in the ER, lacatcystin or H₂O₂) and stress relievers (e.g., cyclohexamide to inhibit protein synthesis) can be used as controls.

Another method contemplated for monitoring the ER stress response is via gene chip analysis. For example, a gene chip with a variety of stress genes can be used to measure expression levels and type of ER stress response (early, late, apoptosis etc.). As one example, the HG-U95A array can be used. (Affymetrix, Inc.).

Lastly, since prolonged ER stress can result in apoptosis and cell death, depending on the level of unfolded proteins in the ER, and the resulting stress level, cells will be more or less sensitive to ER stress inducers such as tunicamycin or proteasome inhibitors. The more sensitive the cells are to the stress inducers, the higher the number of apoptotic or dead cells is observed. Apoptosis can be measured using fluorescent substrates analogs for caspase 3 (an early indicator of apoptosis). FACS, confocal microscopy, and/or using a fluorescence plate reader (96 well format for high through put assays) to determine the percentage of cells positive for apoptosis or cell death (FACS and/or confocal microscopy), or fluorescence intensity can be measured relative to protein concentration in a 96 well format with a fluorescence plate reader.

Another response to cell stress resulting from toxic protein accumulation in the ER is suppression of the ubiquitin/proteasome pathway. This leads to a general disruption of the endocytic pathway (Rocca et al., Molecular Biology of the Cell. 2001; 12:1293-1301). Misfolded protein accumulation is sometimes correlated with increased amounts of polyubiqutin (Lowe et al., Neuropathol Appl Neurobiol. 1990; 16:281-91).

Proteosome function and ubiquitination can be assessed using routine assays. For example, evaluation of 26S proteasome function in living animals by imaging has been achieved ubiquitin-luciferase reporter for bioluminescence imaging (Luker et al., Nature Medicine. 2003. 9, 969-973). Kits for proteasome isolation are commercially available from, for example, Calbiochem (Cat. No. 539176). Ubiquitination can be examined by morphological studies using immunohistochemistry or immunofluorescence. For example, healthy cells and patient cells, treated and untreated, can be fixed and stained with primary antibodies to ubiquitinated proteins and fluorescence detection of secondary antibodies by FACS and/or confocal microscopy will be used to determine changes in ubiquitinated protein levels.

Another assay to detect ubiquitinated proteins is AlphaScreen™ (Perkin-Elmer). In this model, the GST moiety of a GST-UbcH5a fusion protein is ubiquitinated using biotin-Ubiquitin (bio-Ub). Following ubiquitin activation by E1, in the presence of ATP, bio-Ub is transferred to UbcH5a. In this reaction, UbcH5a acts as the carrier to transfer the bio-Ub to its tagged GST moiety. The protein which becomes biotinylated and ubiquitinated is then captured by anti-GST Acceptor and streptavidin. Donor beads resulting in signal generation. No signal will be generated in the absence of ubiquitination.

Lastly, an ELISA sandwich assay can be used to capture ubiquitinated mutant α-Gal A. The primary antibody to the α-Gal A (e.g., rabbit) would be absorbed to the surface, enzyme would be captured during an incubation with cell lysate or serum, then an antibody (e.g., mouse or rat) to ubiquitinated protein, with secondary enzyme-linked detection, would be used to detect and quantify the amount of ubiquitinated enzyme. Alternatively, the assay could be used to quantify the total amount of multi-ubiquitinated proteins in cell extract or serum.

An “improvement in the biomarker” refers to an effect, following treatment resulting in amelioration, reduction, or increase in of one or more biomarkers which are abnormally present, abnormally absent, or present in increased or decreased quantities in patient having lysosomal storage disease relative to a healthy individual who does not have lysosomal storage disease and who does not have another disease that accounts for the abnormal presence, absence, or altered quantities of that surrogate marker.

### Neurophysiological/Neurobehavior Measurements

In some embodiments, the one or more neurophysiological measurements are reliable indicators of lysosomal dysfunction, synaptic dysfunction, synaptic degeneration, microglial activation, astocytosis, neurodegeneration, or combinations thereof. In some embodiments, the one or more neurophysiological measurements comprises gait analysis, neuro-imagining metrics, retinal function, peripheral nerve function, clinical assessment of neurological function, patient-reported episodes, or combinations thereof.

In some embodiments, SubC accumulation is associated with lysosomal storage disease. In some embodiments astrocyte reactivity is measure by the presence of GFAP biomarker. In some embodiments, neurodegeneration is corelated to cortical plate thickness. In some embodiments, neurodegeneration is corelated to calbindin cell counts. In some embodiments, loss of retinal function is corelated to marked atrophy of the nerve fiber and ganglion cell layers. In some embodiments, ERG deficit, such as Light adapted Photopic Response, is corelated with loss of retinal function. In some embodiments, ERG deficit, such as Dark adapted Scotopic Response, is corelated with loss of retinal function.

In some embodiments, the neuro-imagining metrics comprises brain ventricle size, grey matter hyperintensities or hypointensities, white matter hyperintensities or hypointensities, periventricular hyperintensities, cerebellar atrophy, cortical atrophy, whole brain volume, corpus callosum volume, white matter integrity, radial diffusivity, axial diffusivity, magnetic resonance spectroscopy measurements, or combinations thereof.

In some embodiments, the retinal function comprises ERG waveform, visual-evoked potential measurement, optical coherence tomography measurement, or combinations thereof.

In some embodiments, the one or more neurobehavior measurements are reliable indicators of lysosomal dysfunction, synaptic dysfunction, synaptic degeneration, microglial activation, astocytosis, neurodegeneration, or combinations thereof. In some embodiments, the one or more neurobehavioral measurements comprises measuring one or more of: motor function, language function, cognitive function, clinical rating scale, and PROM.

### Tracking Fabry Disease

As non-limiting examples, the disease score for Fabry disease may comprise decreased lysosomal α-Gal A activity in cells (e.g., fibroblasts) and tissue; cellular deposition of GL-3; increased plasma concentrations of homocysteine and vascular cell adhesion molecule-1 (VCAM-1); GL-3 accumulation within myocardial cells and valvular fibrocytes, leading to cardiac hypertrophy (especially of the left ventricle), valvular insufficiency, and arrhythmias; proteinuria; increased urinary concentrations of lipids such as CTH, lactosylceramide, ceramide, and decreased urinary concentrations of glucosylceramide and sphingomyelin (Fuller et al., Clinical Chemistry. 2005; 51:688-694); the presence of laminated inclusion bodies (Zebra bodies) in glomerular epithelial cells; renal failure; hypohidrosis (which causes heat intolerance); the presence of angiokeratomas; and hearing abnormalities such as high frequency sensorineural hearing loss progressive hearing loss, sudden deafness, or tinnitus. Neurological symptoms include transient ischemic attack (TIA) or stroke; and neuropathic pain manifesting itself as acroparaesthesia (burning or tingling in extremities).

Globotriaosylceramide accumulation. A method for measuring globotriaosylceramide (GB₃, or GL-3) levels in plasma and urine of humans affected by lysosomal storage disease, such as Fabry disease, is described in, e.g., Boscaro et al., Rapid Commun Mass Spectrom. 2002; 16(16):1507-14. In this reference, the analyses are performed using flow injection analysis-electrospray ionization-tandem mass spectrometry (FIA-ESI-MS/MS).

Immunoelectron-microscopic detection of GL-3 accumulated in the skin of patients with Fabry disease has been described in Kanekura et al., Br J Dermatol. 2005; 153(3):544-8. This method is sensitive enough to detect lysosomal accumulation of GL-3. Skin biopsies can be obtained by using a “punch” device, which removes a sample layer of skin.

Renal biopsies are performed using ultrasound, x-ray or CT scan guidance. Under some circumstances, the biopsy is be performed by running the biopsy catheter through one of the neck veins-this is called a trans jugular biopsy. GL-3 accumulation in kidney, specifically in all renal cell types, including vascular endothelial cells, vascular smooth muscle cells, mesangial cells and interstitial cells, podocytes and distal tubular epithelial cells, had been described in Thurburg et al., Kidney Int. 2002; 62(6):1933-46. Ultrastructural study (electron microscopy) of kidney biopsies can reveal typical inclusion bodies in the cytoplasm of all types of renal cells (Sessa et al., J Inherit Metab Dis. 2001; 24 Suppl 2:66-70). The cells are characterized by concentric lamellation of clear and dark layers (“zebra” or “onion-skin” appearance) with a periodicity of 35-50 A.

Kidney function can be assessed by determining glomerular filtration rate (ml/min) and by assessing serum creatine levels according to well-established methods. Other renal assessments include 24-hour protein excretion, urine protein electrophoresis, total protein, microalbumin, urine beta-2 microglobulin titers. Reduction in GL-3 sediment and proteinuria is a direct measurement of renal health.

Recently, atmospheric pressure photoionization mass spectrometry (APPI-MS) was shown to be an efficient method for the analysis of GL-3 molecular species, both in direct injection and by coupling with liquid chromatography (LC). This technique allowed the detection of a great number of species from biological samples isolated from Fabry patients (Delobel et al., J Mass Spectrom. 2005 Nov. 14; [Epub ahead of print]).

α-Galactosidase activity. As indicated above, non-invasive assessment of α-Gal A activity can be measured in blood leukocytes or in cultured fibroblasts from skin biopsies. Such assays typically involve extraction of blood leukocytes from the patient, lysing of the cells, and determining the activity in the lysate upon addition of an enzyme substrate such as 4-methyl umbelliferal alpha-D-galactoside an/or N-acetylgalactosamine (see U.S. Pat. No. 6.274,597).

Two sensitive immunoassays for the measurement of α-Gal A activity and protein to determine the concentrations of alpha-galactosidase in blood and plasma are described in Fuller et al., Clin Chem. 2004; 50(11): 1979-85.

Cardiac evaluation. Increases in alpha-Gal A activity may play a role monitoring or detecting heart disease or in at least a subset of heart disease patients. Evaluation of GL-3 in cardiac cells can be achieved through endomyocardial biopsies. This is an invasive procedure that involves using a bioptome (a small catheter with a grasping device on the end) to obtain a small piece of heart muscle tissue.

GL-3 present in perinuclear vacuoles will stain positive with an acid stain. In addition, histological examination of the biopsies can be done using transmission electron microscopy to ascertain thickening of endocardium to measure ventricular mass, or to determine the presence of hypertrophic myocardial fibers.

In addition, common carotid and radial artery diameter, intima-media thickness (IMT) and distensibility have been assessed using high-definition echotracking systems and aplanation tonometry. (Boutouyrie et al., Acta Paediatr Suppl. 2002; 91(439):62-6. Cardiac myocytes will also be examined for accumulation of GL-3. Macroscopic cardiac morphology can be assessed using MRI or Doppler echocardiography. Cardiac function can be assessed by, e.g., determining left ventricular ejection fraction and using electrocardiograms.

Neuropathic pain/peripheral neuropathy. Pain in the extremities can be assessed using subjective tests given to the patient. In addition, to evaluate neuropathy, Quantitative Sensory Testing (CASE study) can be used. A CASE study is a biophysical technique in which a patient is asked to push a button as soon as he feels either a sensation of cold, warmth, or vibration. These stimulations are delivered by an electrode that is put on the skin of the hand or foot.

Cerebrovascular. In addition to stroke and hypertension, other Fabry-related cerebrovascular signs and symptoms associated can include hemiparesis, vertigo, double vision; seizures; basilar artery ischemia and aneurism; labyrinthine disorders or cerebral hemorrhage.

Neurological. In male and female patients with lysosomal storage disease, such as Fabry disease, significant age-related cerebral white matter lesions (WMLs) can be found. Evaluation of neurological effects can be assessed using, e.g., Quantitative Sudomotor Axon Reflex Test (QSART). QSART is a routine test of autonomic function and a sensitive test in distal small-fiber neuropathy such as observed in Fabry disease.

Hypohydrosis/anhidrosis. Impaired sweating and heat intolerance in patients diagnosed with lysosomal storage disease, such as Fabry disease, has been attributed to selective peripheral nerve damage or to intracytoplasmic lipid deposits in the small blood vessels surrounding sweat glands.

Hilz et al. (i Acta Paediatr Suppl. 2002; 91(439):38-42) have described the methods to assess impairment of temperature perception, vibratory perception, sudomotor and eccrine sweat gland function, and limb and superficial skin blood flow and vasoreactivity in patients diagnosed with lysosomal storage disease, such as Fabry disease. These methods include thermal provocation tests, quantitative sudomotor axon reflex testing (QSART) and venous occlusion plethsmography. QSART has three parts and measures resting skin temperature, resting sweat output, and stimulated sweat output. Measurements are typically taken on arms, legs or both. A small plastic cup is placed on the skin and the temperature and amounts of sweat under the skin are measured. To stimulate sweat a chemical is delivered electrically through the skin to a sweat gland, but the patient will only feel warmth. A computer is used to analyze the data to determine how well the nerves and sweat glands are functioning.

In addition, a reduction of tears and saliva is also observed in some patients having lysosomal storage disease. For example, about 40% of Fabry disease patients experience reduction of tears and saliva.

Temperature intolerance. In addition to heat intolerance, cold and heat sensitivity often results from lipid deposition in small vessel walls, perineural cells, and unmyelinated or myelinated nerve cells resulting in small fiber neuropathy.

Ophthalmologic opacities. Patients diagnosed with lysosomal storage disease may exhibit whorled corneal opacities, lenticular opacities, and vascular lesions of the conjunctivae and retina. Corneal opacities can be seen using slit lamp microscopy. In Fabry patients, two types of lens opacities have been noted: cream-colored anterior capsular deposits in the lens (sometimes distributed like a propeller), and whitish, granular spoke-like deposits on the posterior lens (referred to as Fabry cataracts).

Hearing loss. Non-invasive methods to evaluate cochlear functions using conventional audiometry, tympanometry, ABR audiometry, and otoacoustic emissions is described in Germain et al., BMC Med Genet. 2002; 3(1):10).

Gastrointestinal disturbance. Gastrointestinal symptoms may result from deposition of glycosphingolipids in mesenteric blood vessels and autonomic ganglia. Symptoms include postprandial bloating; abdominal cramping and pain; early satiety; diarrhea; constipation; nausea; vomiting.

Other surrogate markers. Other markers of Fabry disease include Lymphoedema (swelling of the extremities) due to accumulation of GL-3. In addition, it was recently discovered that there was a significant decrease in diastolic blood pressure in patients with lysosomal storage disease, such as Fabry disease, which may account for exercise tolerance (Bierer et al., Respiration. 2005; 72(5):504-11).

### Treating Lysosomal Storage Disease

Another aspect of the disclosure relates to method of treating lysosomal storage disease (LSD) in a patient. In some embodiment, the method comprising determining a disease score of the patient, and administering the patient a therapy. In some embodiments, the therapy comprises an enzyme replacement therapy, a gene therapy and a small molecule.

A “responder” is an individual diagnosed with a lysosomal storage disease and treated and monitored according to the presently claimed method, who exhibits an improvement in one or more surrogate markers, and/or amelioration of, or reversal of, disease progression.

In addition, a determination whether an individual is a responder can be made at the sub-cellular level by evaluating, e.g., intracellular trafficking of a therapeutic protein in response to a treatment. Restoration of trafficking from the ER to the lysosome is indicative of a response. Other sub-cellular evaluations that can be assessed to determine if an individual is a responder include improvements in the above-referenced sub-cellular surrogate markers.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

In some embodiments, the therapy comprises enzyme replacement therapy (ERT). The ERT typically involves intravenous infusion of a purified form of a therapeutic protein. In some embodiments, the therapeutic protein corresponds to wild-type protein or a mutant thereof. In some embodiments, the therapeutic protein comprises palmitoyl protein thioesterase 1 (PPT1) (CLN1), tripeptidyl peptidase 1 (TPP1) (CLN2), Cathepsin D (CTSD) (CLN10), progranulin (PGRN) (CLN11) and cathepsin F (CTSF) (CLN13), alpha-galactosidase A, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, or an enzymatically active fragment thereof. In one or more embodiments, the therapeutic protein comprises a Batten-related protein selected from PPT1, TPP1, CTSD, PGRN or CTSF.

In some embodiments, the therapeutic protein is a Batten-related protein. In one or more embodiments, the therapeutic protein comprises a ceroid-lipofuscinosis neuronal protein. In one or more embodiments, the therapeutic protein comprises ceroid-lipofuscinosis neuronal protein 1, ceroid-lipofuscinosis neuronal protein 2, ceroid-lipofuscinosis neuronal protein 3, ceroid-lipofuscinosis neuronal protein 4, ceroid-lipofuscinosis neuronal protein 5, ceroid-lipofuscinosis neuronal protein 6, ceroid-lipofuscinosis neuronal protein 7, ceroid-lipofuscinosis neuronal protein 8,, ceroid-lipofuscinosis neuronal protein 10, ceroid-lipofuscinosis neuronal protein 11, ceroid-lipofuscinosis neuronal protein 12, ceroid-lipofuscinosis neuronal protein 13, or ceroid-lipofuscinosis neuronal protein 14.

In some embodiments, the therapeutic protein is an α-Gal A protein. Two α-Gal A products are currently available for the treatment of Fabry disease: agalsidase alfa (Replagal®, Shire Human Genetic Therapies) and agalsidase beta (Fabrazyme®; Sanofi Genzyme Corporation).

In one or more embodiments, the therapy comprise gene therapy. In some embodiments, the gene therapy is used for delivering a transgene to the patient. In some embodiments, the transgene encodes the therapeutic protein. In some embodiments, rAAV is used for delivering the transgene.

Adeno-associated virus (AAV) is a replication-deficient parvovirus, the single-stranded DNA genome of which is about 4.7 kb in length including 145 nucleotide inverted terminal repeat (ITRs) and may be used to refer to the virus itself or derivatives thereof. The term covers all subtypes and both naturally occurring and recombinant forms, except where specified otherwise. There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

rAAV provided herein can comprise a polynucleotide sequence that encodes a polypeptide with CLN3 activity and that hybridizes under stringent conditions to the nucleic acid sequence of SEQ ID NO:2, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

The rAAV genomes comprise one or more AAV ITRs flanking the polynucleotide encoding the therapeutic protein. The transgene is operatively linked to transcriptional control elements (including, but not limited to, promoters, enhancers and/or polyadenylation signal sequences) that are functional in target cells to form a gene cassette. Examples of promoters are the P546 promoter and the chicken β actin promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Provided herein are a P546 promoter sequence set out in SEQ ID NO: 3, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 3 that are promoters with P546 transcription promoting activity. Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Examples of inducible promoters include, but are not limited to a metallothionine promoter, a glucocorticoid promoter, a progesterone promoter, and a tetracycline-regulated promoter. The gene cassette may also include intron sequences to facilitate processing of a therapeutic protein RNA transcript when expressed in mammalian cells. One example of such an intron is the SV40 intron.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes disclosed herein may lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014).

DNA plasmids disclosed herein comprise rAAV genomes described herein. The DNA plasmids are transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV particles, in which an AAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

Further disclosed herein are packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also disclosed herein are rAAV (i.e., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. An rAAV with a single-stranded genome is referred to herein as an ssAAV.

An exemplary scAAV provided herein is the rAAV named “scAAV9.P546.CLN3.” The scAAV9.P546.CLN3 rAAV comprises a human CLN3 cDNA under the control of a truncated Methyl CpG binding protein 2 (MeCP2) promoter herein referred to as the P546 promoter (SEQ ID NO: 3). The CLN3 cDNA has a polynucleotide sequence set out in SEQ ID NO: 1. The SEQ ID NO:1 encodes a polypeptide sequence provided in SEQ ID NO:2. The rAAV also comprises a SV40 Intron (upstream of human CLN3 cDNA) and Bovine Growth Hormone polyadenylation (BGH Poly A) terminator sequence (downstream of human CLN3 cDNA). The sequence of this scAAV9.P546.CLN3 gene cassette is set out in SEQ ID NO: 4. The rAAV is packaged in AAV9 capsid and includes AAV2 ITRs (one ITR upstream of the P546 promoter and the other ITR downstream of the BGH Poly A terminator sequence).

The rAAV may be purified by methods standard in the art such as by column chromatography or cesium chloride gradients. Methods for purifying rAAV from helper virus are known in the art and include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions comprise a rAAV encoding the therapeutic protein. Compositions may include two or more rAAV encoding different therapeutic proteins of interest.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20% to about 40% non-ionic, low-osmolar compound or about 25% to about 35% non-ionic, low-osmolar compound. An exemplary composition comprises rAAV in 20 mM Tris (pH8.0), 1 mM MgCl₂, 200 mM NaCl, 0.001% poloxamer 188 and about 20% to about 40% non-ionic, low-osmolar compound.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include from about 1×10¹¹, about 1×10¹², about 1×10¹³, about 6×10¹³, about 1×10¹⁴, about 2×10¹⁴, about 3×10¹⁴, about 4×10¹⁴, about 5×10¹⁴, about 1×10¹⁵, to about 1×10¹⁶, or more total viral genomes. Dosages of about 1×10¹² to about 1×10¹⁵ vg, about 1×10¹³ to about 6×10¹⁴ vg, about 6×10¹³ to about 1.2×10¹⁴ vg and about 2×10¹⁴ vg to about 4×10¹⁴ vg are also contemplated. One dose exemplified herein is 6×10¹³ vg. Another dose exemplified herein is 1.2×10¹⁴ vg.

Methods of transducing target cells (including, but not limited to, nerve or glial cells) with rAAV are provided. The cells of the nervous system include neurons, lower motor neurons, microglial cells, oligodendrocytes, astrocytes, Schwann cells or combinations thereof.

The term “transduction” is used to refer to the administration/delivery of the transgene to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. In some embodiments, the gene therapy is administered/delivered systemically or to central nervous system. In some embodiments, the gene therapy is administered/delivered to a brain. In some embodiments, the gene therapy is administered/delivered to a spinal cord. In some embodiments, the patient is administered/delivered rAAV encoding the therapeutic protein by an intrathecal, intracerebroventricular, intraparechymal, or intravenous route, or any combination thereof. Intrathecal delivery refers to delivery into the space under the arachnoid membrane of the brain or spinal cord. In some embodiments, intrathecal administration is via intracisternal administration. In some embodiments, intrathecal administration is via intra cisterna magna (ICM) administration. In some embodiments, the intra cisterna magna (ICM) administration is at the craniocervical junction.

Intrathecal administration is exemplified herein. The therapy comprises transducing target cells (including, but not limited to, nerve and/or glial cells) with one or more rAAV described herein. In some embodiments, the rAAV viral particle comprising a polynucleotide encoding the therapeutic protein is administered or delivered to the brain and/or spinal cord of a patient. In some embodiments, the polynucleotide is delivered to brain. Areas of the brain contemplated for delivery include, but are not limited to, the motor cortex and the brain stem. In some embodiments, the polynucleotide is delivered to the spinal cord. In some embodiments, the polynucleotide is delivered to a lower motor neuron. In some embodiments, the polynucleotide is delivered to nerve and glial cells. In some embodiments, the glial cell is a microglial cell, an oligodendrocyte or an astrocyte. In some embodiments, the polynucleotide is delivered to a Schwann cell.

In methods provided herein, the patient can be held in the Trendelenberg position (head down position) after administration of the rAAV (e.g., for about 5, about 10, about 15 or about 20 minutes). For example, the patient is tilted in the head down position at about 1 degree to about 30 degrees, about 15 to about 30 degrees, about 30 to about 60 degrees, about 60 to about 90 degrees, or about 90 to about 180 degrees).

In one or more embodiments, the therapy comprises use of small molecule inhibitors to reduce production of the natural substrate of deficient enzyme proteins, thereby ameliorating the pathology. This “substrate reduction” approach has been specifically described for a class of about 40 LSDs that include glycosphingolipid storage diseases. The small molecule inhibitors proposed for use as therapy are specific for inhibiting the enzymes involved in synthesis of glycolipids, reducing the amount of cellular glycolipid that needs to be broken down by the deficient enzyme.

In one or more embodiments, the therapy comprises use of pharmacological chaperones (PCs). Such PCs include small molecule inhibitors of enzymes, which can bind to the enzyme to increase the stability of both mutant enzyme and the corresponding wild type.

Combination therapies are also provided. Combination as used herein includes either simultaneous treatment or sequential treatment. Combinations of methods described herein with standard medical treatments are specifically contemplated.

While delivery to a subject in need thereof after birth is contemplated, intrauterine delivery to a fetus is also contemplated.

The present invention also provides a method for monitoring the treatment of patients having lysosomal storage disease with specific pharmacological chaperones. Specifically, various assays are employed to evaluate the progress of the disease and its response to treatment. In particular, various systemic and sub-cellular markers can be assayed. The monitoring aspect of the present invention encompasses both invasive and non-invasive measurement of various cellular substances.

### EXAMPLES

While the following examples describe specific embodiments, it is understood that variations and modifications will occur to those skilled in the art. Accordingly, only such limitations as appear in the claims should be placed on the invention.

### Example 1: Disease Score Model for CLN3

The purpose of this ongoing study is to build a longitudinal disease score model for Cln3 disease using biofluid biomarker and neurophysiological/neurobehavioral measurements from Cln3^{Δex7/8} pigs. The disease scoring system could subsequently provide a sensitive outcome-monitoring platform for testing therapeutics in this model, and could have translational value due to the direct clinical correlates for every assay used in the development of the score.

### Identification of Biomarkers in Cln3^{Δex7/8} Pigs

A longitudinal cohort of Cln3^{Δex7/8} and wild type pigs is being used for this study, which includes metabolomic and proteomic profiling. The metabolomic and proteomic profiling was performed in wildtype (WT) and Cln3^{Δex7/8} pigs according to Table 1.

TABLE 1
Study design for analysis of biomarker
levels in Cln3^{Δex7/8} pig serum

N

| | 6 | 24-27 | 36 | 47-50 |
|---|---|---|---|---|
| | Month | Month | Month | Month |
| WT | 6 | 9 | 10 | 3 |
| Cln3^{Δex7/8} | 6 | 9 | 10 | 3 |

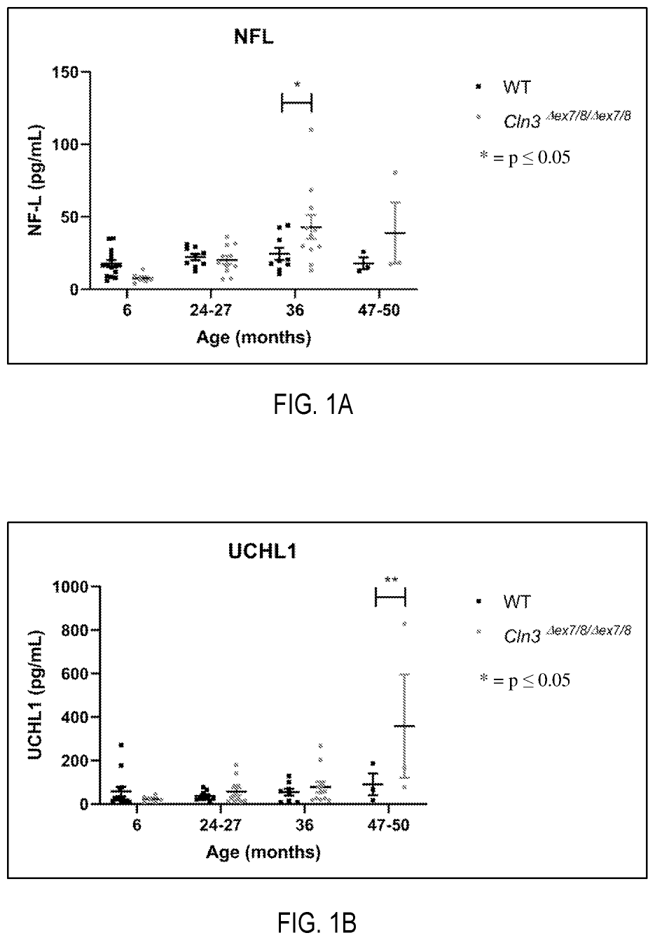

As part of our discovery profiling, a targeted analysis for neurofilament light (NFL), a high-potential biomarker target, was conducted. NFL is a cytoskeletal protein specific to neurons that has emerged as a valuable biomarker for neuronal damage, with utility in conditions ranging from traumatic brain injury to Alzheimer's disease. NFL has also been shown to be elevated in CLN2 and CLN3 disease patients^{7, 8}, and to respond to enzyme replacement therapy in CLN2 patients⁷. FIG. 1A shows our analysis revealing longitudinal increases in CLN3 human patients. NFL was measured in CLN3 human serum samples from four time points using the Quanterix SIMOA platform.

FIG. 1B shows our analysis revealing longitudinal increase in NFL in Cln3^{Δex7/8} pigs. NFL was measured on WT and Cln3^{Δex7/8} pig serum samples from four time points using the Quanterix SIMOA platform. Significant (p<0.05) elevations were observed at 36 months of age. Two-way ANOVA with Tukey's post-hoc test was performed to determine degree of elevation. At least 38 species were observed to be significantly upregulated in CLN3^{Δex7-8} samples.

The degree of elevation observed was remarkably similar to that observed in human CLN3 patients⁸, confirming the utility of the Cln3^{Δex7/8} pig model for biomarker development.

Additionally, on a group level, NFL is significantly elevated at 36 months of age, with trends towards elevations at 47-50 months of age (note: only n=3 animals for each genotype were available at this late time point). Due in part to the substantial overlap in the levels observed between groups, however, NFL will likely be of limited value as a clinical biomarker.

Similar to NFL, another marker of neurodegeneration, ubiquitin c-terminal hydrolase L1 (UCHL1), was also analyzed. FIG. 1C shows our analysis revealing longitudinal increases in Cln3^{Δex7/8} pigs. UCHL1 was measured on WT and Cln3^{Δex7/8} pig serum samples from four time points using the Quanterix SIMOA platform. Significant (p<0.05) elevations were observed at 36 months of age. Two-way ANOVA with Tukey's post-hoc test was performed to determine degree of elevation.

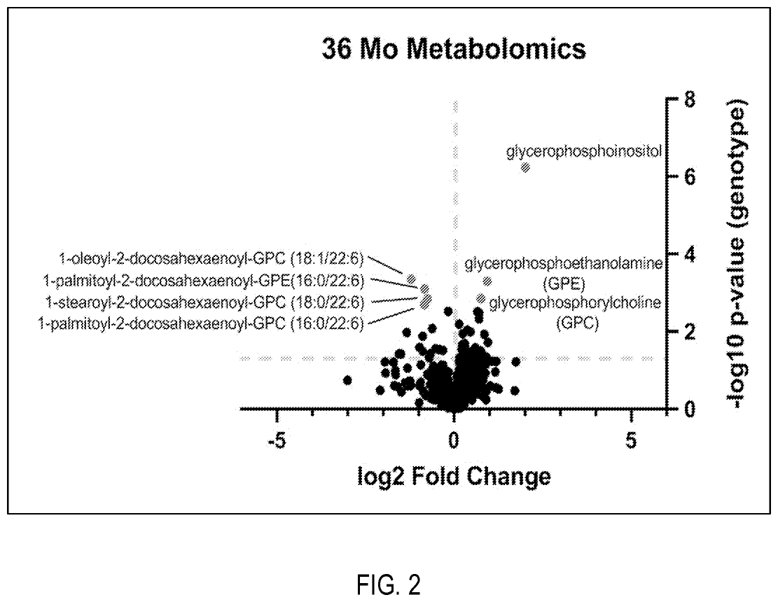

In additional to NFL and UCHL1, our metabolomic profiling has revealed several candidate biomarkers with far greater separation between WT and Cln3^{Δex7/8} pigs. Of the 805 targets, which includes 350 lipids and 451 small molecule metabolites, quantitatively analyzed in metabolomics profiling. In FIG. 2, each target was plotted with log? Fold Change on the x-axis and −log₁₀ p-value (genotype difference) on the y-axis. Relative potential for each target as a biomarker was correlated with distance from the origin on the graph. Highest potential candidates were denoted by grey round points on the graph. Targets above the horizontal dashed grey line are significantly (p<0.05) different between genotypes. The vertical dashed grey line signifies a fold change of “0”. An analysis of FIG. 2 indicates a small subset of glycerophospho metabolites and related lipid-conjugated species are substantially changed in Cln3^{Δex7/8} pigs.

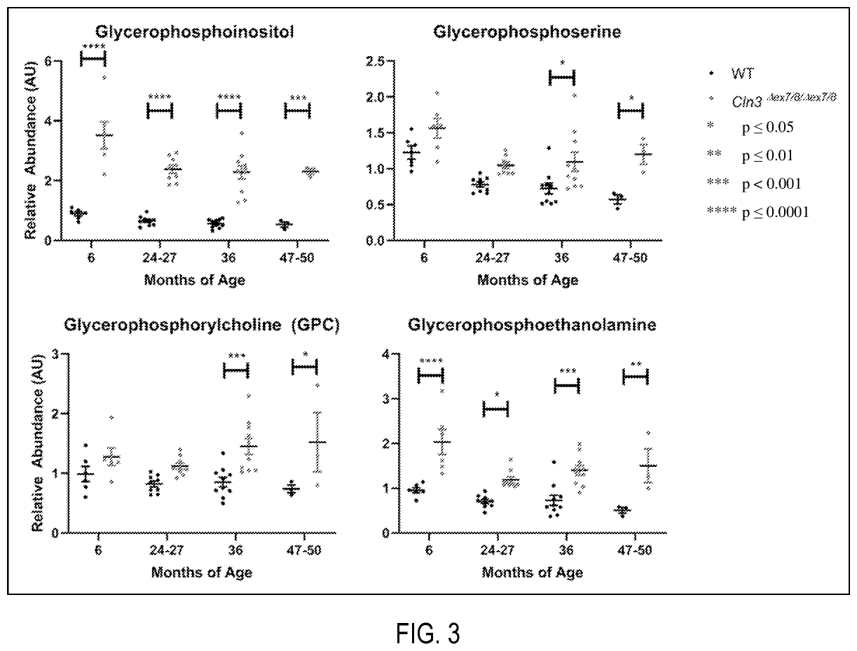

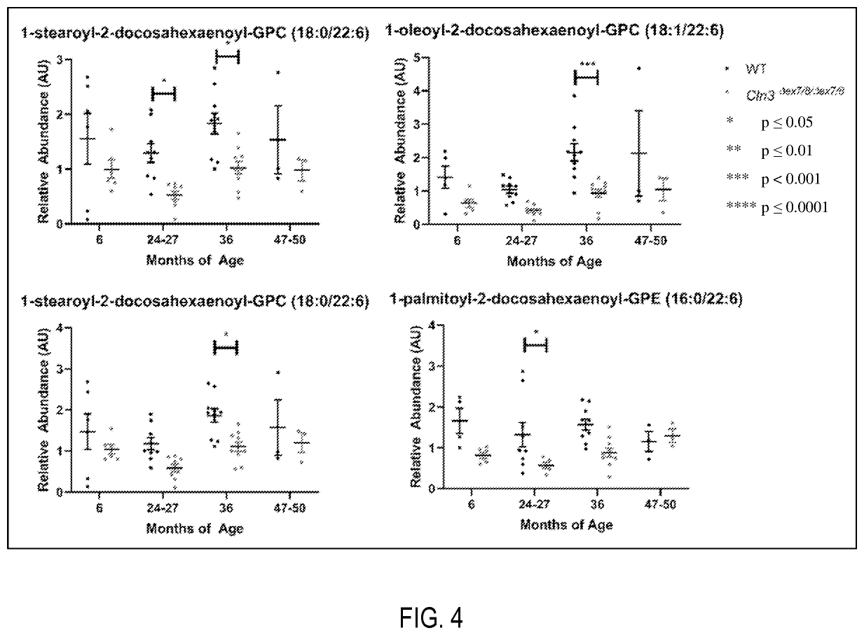

FIG. 3 shows relative levels of glycerophosphoinositol (upper left), glycerophosphoserine (upper right), glycerophosphorylcholine (lower left), and glycerophosphoethanolamine (lower right) in WT and Cln3^{Δex7/8} pigs at 6, 24-27, 36, and 47-50 months of age. Similarly, FIG. 4 shows relative levels of 1-stearoyl-2-docosahexaenoyl-GPC 18:0/22:6 (upper left), 1-palmitoyl-2-docosahexaenoyl-GPE 16:0/22:6 (upper right), 1-stearoyl-2-docosahexaenoyl-GPC 18:0/22:6 (lower left), and 1-oleoyl-2-docosahexaenoyl-GPC 18:1/22:6 (lower right) in WT and Cln3^{Δex7/8} pigs at 6, 24-27, 36, and 47-50 months of age. Two-way ANOVA with Tukey's post-hoc test were performed to determine degree of elevation.

An analysis of FIG. 2 and FIG. 3 revealed that glycerophosphoinositol exhibited by far the most significant difference between genotypes. Similarly, glycerophosphoethanolamine (GPE), glycerophosphoserine, and glycerophosphorylcholine (GPC) also exhibited significant elevations.

An analysis of FIG. 2 and FIG. 4 revealed that several docosahexaenoic acid (DHA) conjugates of GPE and GPC were significantly lower in Cln3^{Δex7/8} pigs.

### Validation of Biomarkers in Cln3^{Δex7/8} Pigs

The biomarkers can be validated by analyzing the samples on a similar platform with Creative Proteomics that was used for identification of biomarkers. The validation can be based upon absolute, rather than relative, quantitation for glycerophosphoinositol, glycerophosphorylcholine, glycerophosphoserine, and glycerophosphoethanolamine.

Given the large decreases in free DHA and DHA phospholipids in Cln3^{Δex7/8} pig serum, it is likely that levels of DHA-derived eicosanoids are also altered, perhaps to an even greater extent. A comprehensive investigation into eicosanoid levels in CLN3^{Δex7/8} pig serum can be conducted by quantifying absolute levels of eicosanoids in samples using a liquid chromatography with tandem mass spectrometry (LC MS/MS) based approach. Biomarker candidates can be identified among the eicosanoids identified, and all data points can be combined with those from our comprehensive metabolomic, lipidomic, and proteomic profiling to enhance the power of our machine-learning based disease score modeling.

The biomarkers identified and/or validated in this study can be used for monitoring disease progression and therapeutic response, and can greatly enhance the power of the disease score model that is currently being developed. Upon validation in human patient samples, biomarkers identified through this study could be immediately adopted into clinical trial designs for CLN3 disease.

### Neurophysiological/Neurobehavior Measurement in Cln3^{Δex7-8/Δex7-8} Pigs

85% of patients diagnosed with CLN3 encompasses exons 7 to 8, resulting in a truncated, nonfunctional protein. Accordingly, rAAV-mediated gene targeting was used to introduce the CLN3 mutation in fibroblasts. Somatic cell nuclear transfer, reconstructed embryos were transferred to recipient pigs. Heterozygote progenitor pigs were then bred to expand the colony and generate homozygote Cln3^{Δex7-8/Δex7-8} Pigs.

Brain sections including VPM-VPL, CA2-CA3, motor cortex and somatosensory cortex were collected from WT and Cln3^{Δex7-8/Δex7-8} pigs at 2, 6, 14, 36 and 48 months of age. The sections were immune-stained and images were captured. The images were processed to determine immunoreactivity as a function of the % area.

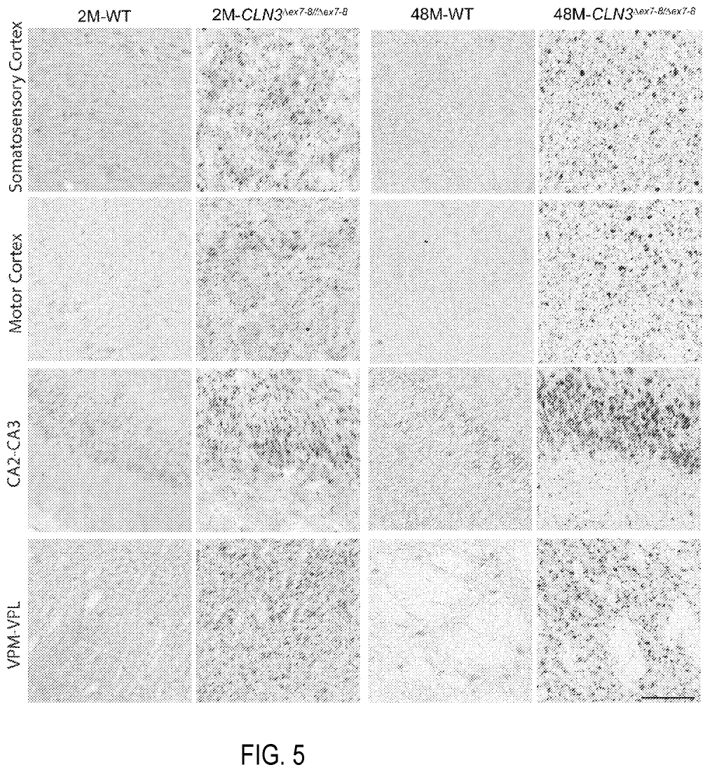

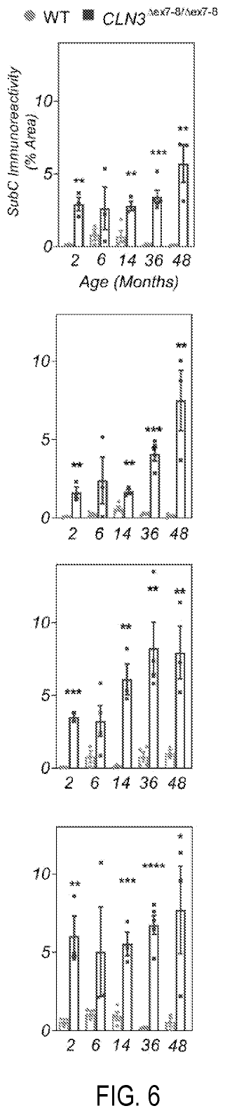

All sections from VPM-VPL, CA2-CA3, motor cortex and somatosensory cortex were processed to determine early and/or persistent SubC accumulation. FIG. 5 shows representative images of sections from WT and Cln3^{Δex7-8/Δex7-8} pigs at 2 and 48 months of age that are immune-stained for SubC. FIG. 6 shows an analysis of SubC immunoreactivity in VPM-VPL, CA2-CA3, motor cortex and somatosensory cortex of WT and Cln3^{Δex7-8/Δex7-8} pigs at 2, 6, 14, 36 and 48 months of age.

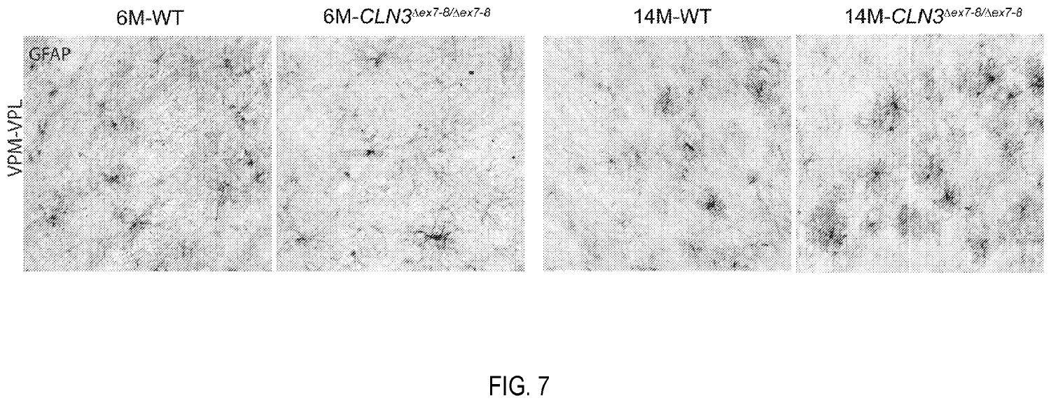

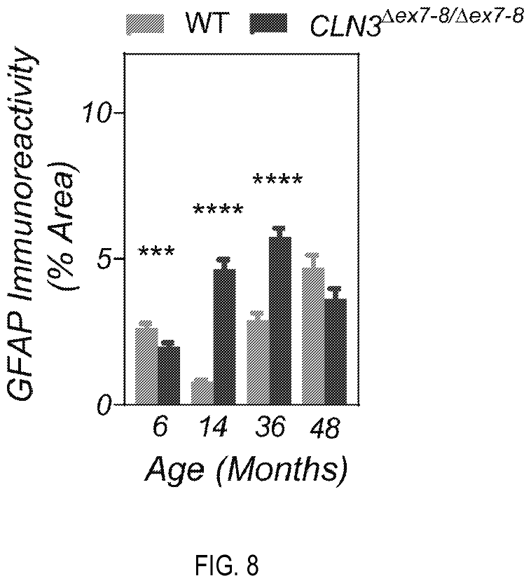

Similarly, VPM-VPL sections were processed to determine astrocyte reactivity. GFAP a biomarker for determining astrocyte reactivity. FIG. 7 shows representative images of VPM-VPL section from WT and Cln3^{Δex7-8/Δex7-8} pigs at 6 and 14 months of age that are immune-stained for GFAP. FIG. 8 shows an analysis of GFAP immunoreactivity at 6, 14, 36 and 48 months of age. An analysis revealed that Cln3^{Δex7-8/Δex7-8} pigs gain astrocyte reactivity by 14 months of age.

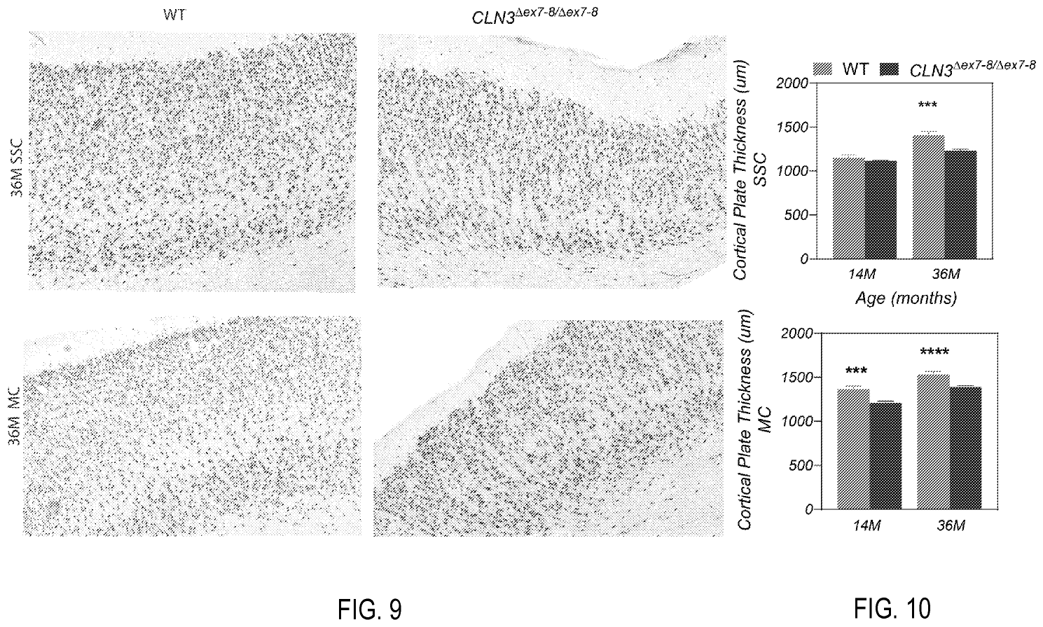

Motor cortex and somatosensory cortex sections were processed to determine cortical neurodegeneration. FIG. 9 shows representative images of motor cortex and somatosensory cortex sections from WT and Cln3^{Δex7-8/Δex7-8} pigs at 36 months of age that were used to determine cortical plate thickness. FIG. 10 shows cortical plate thickness of motor cortex and somatosensory cortex sections from WT and Cln3^{Δex7-8/Δex7-8} pigs at 14 and 36 months of age.

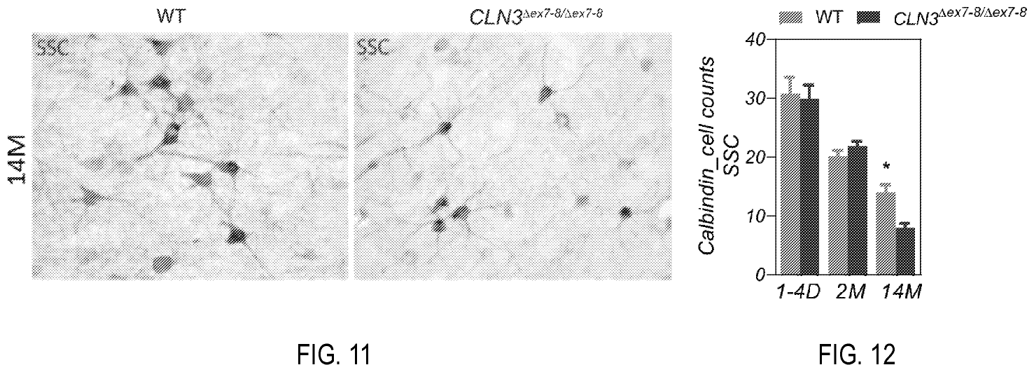

Somatosensory cortex sections were processed to determine cortical neurodegeneration using immune-staining for calbindin. FIG. 11 shows representative images of somatosensory cortex sections from WT and Cln3^{Δex7-8/Δex7-8} pigs at 14 months of age that were immune-stained for calbindin. FIG. 12 shows an analysis of calbindin cell counts at 1-4 days, 2 months and 14 months of age.

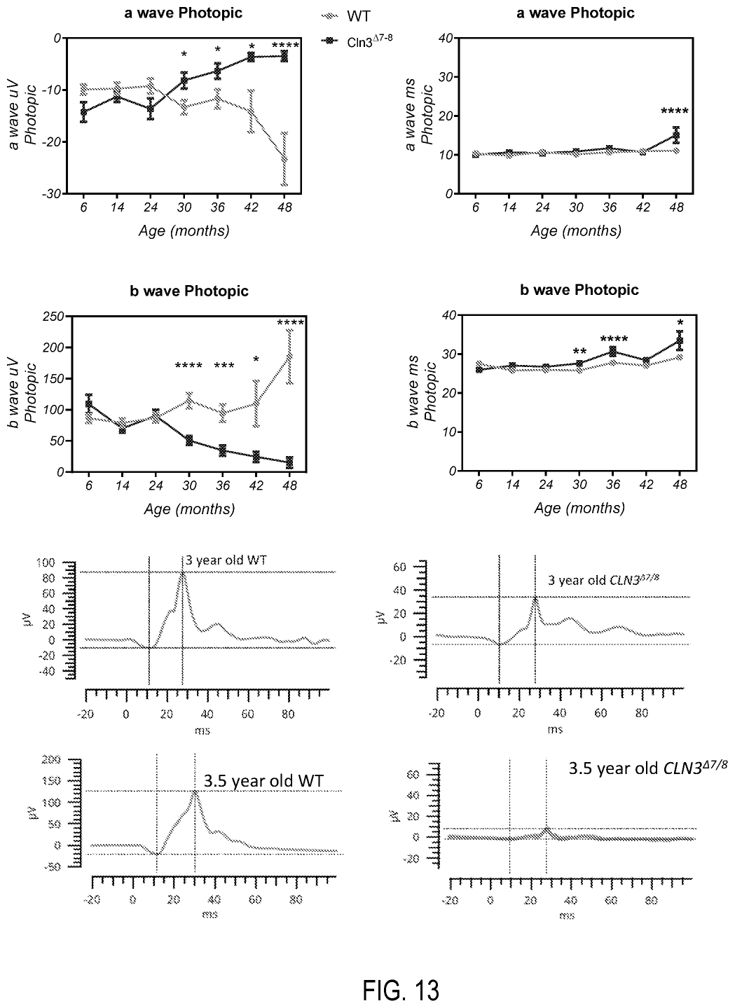

Electroretinogram (ERG) were also measured for WT and Cln3^{Δex7-8/Δex7-8} pigs at 6, 14, 24, 30, 36, 42, and 48 months of age. The results are shown in FIG. 13 indicating ERG deficits are observed by 30 months of age.

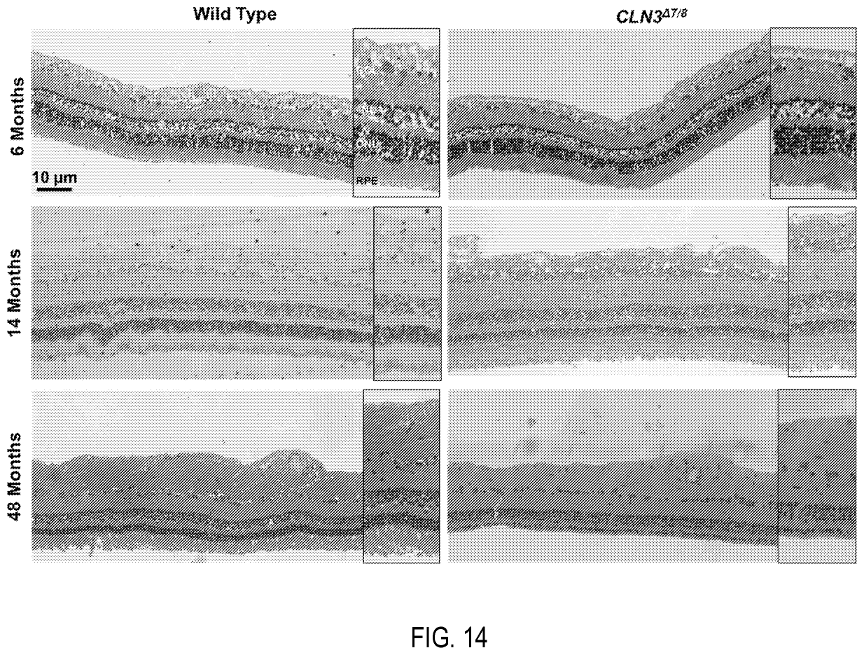

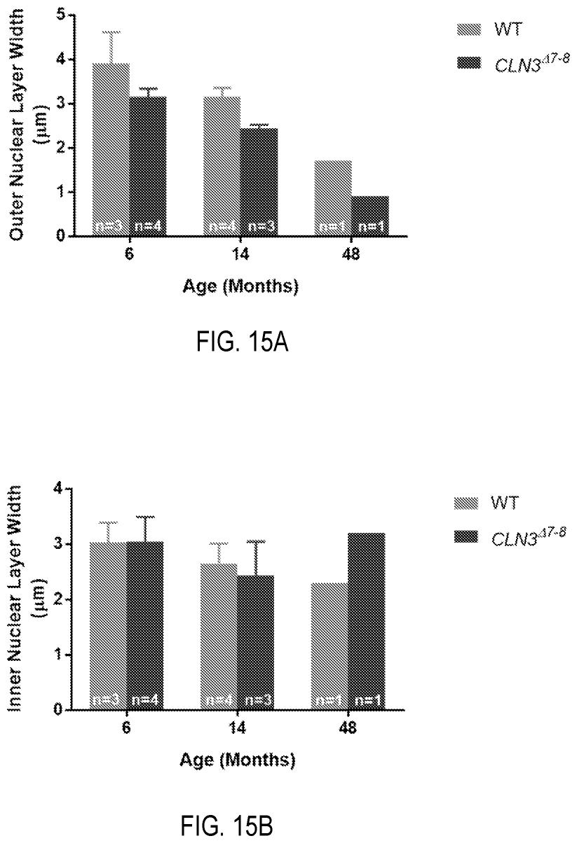

CLN3 patients lose in Ganglion cell layer (GCL) with retinal ganglion cells. Accordingly, retina sections of WT and Cln3^{Δex7-8/Δex7-8} pigs at 6, 14, and 48 months of age were analyzed as shown in FIG. 14. FIG. 15 shows an analysis of outer nuclear layer width and inner nuclear layer width in WT and Cln3^{Δex7-8/Δex7-8} pigs at 6, 14, and 48 months of age.

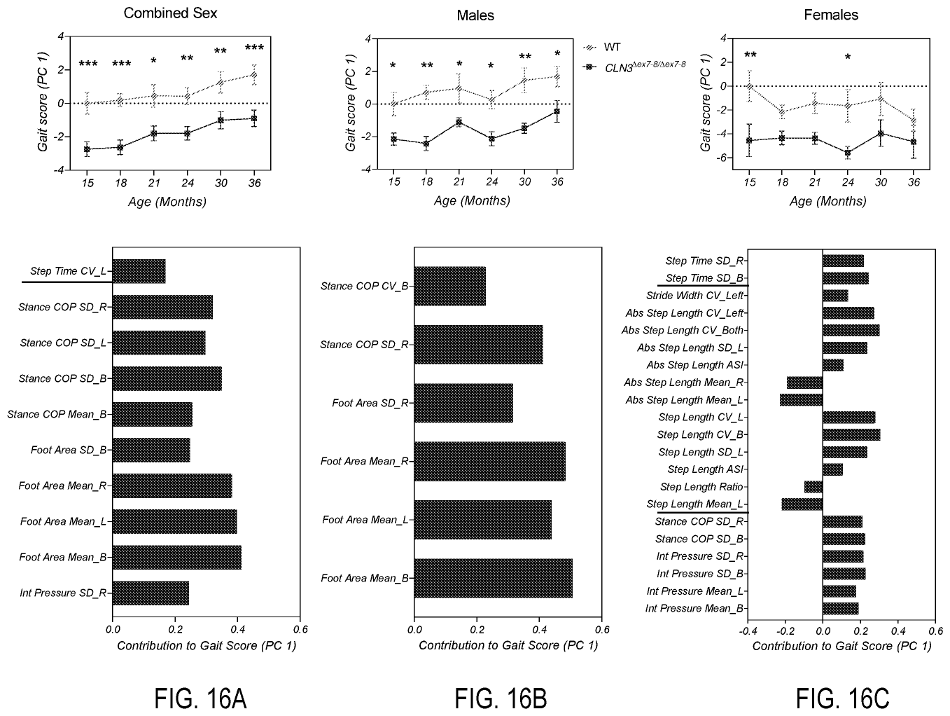

Gait was measured for WT and Cln3^{Δex7-8/Δex7-8} pigs at 15, 18, 21, 24, 30, and 36 months of age using Zeno Electronic Sensor Mat. Animals walked normally on the mat several times, and 8 consecutive steps from the front legs were analyzed from each ‘walk.” 39 parameters were used for determining principal component analysis, the results of which are shown in FIG. 16.

### Example 2: Identifying Potential Biofluid Biomarker for CLN6 and CLN8 Disease

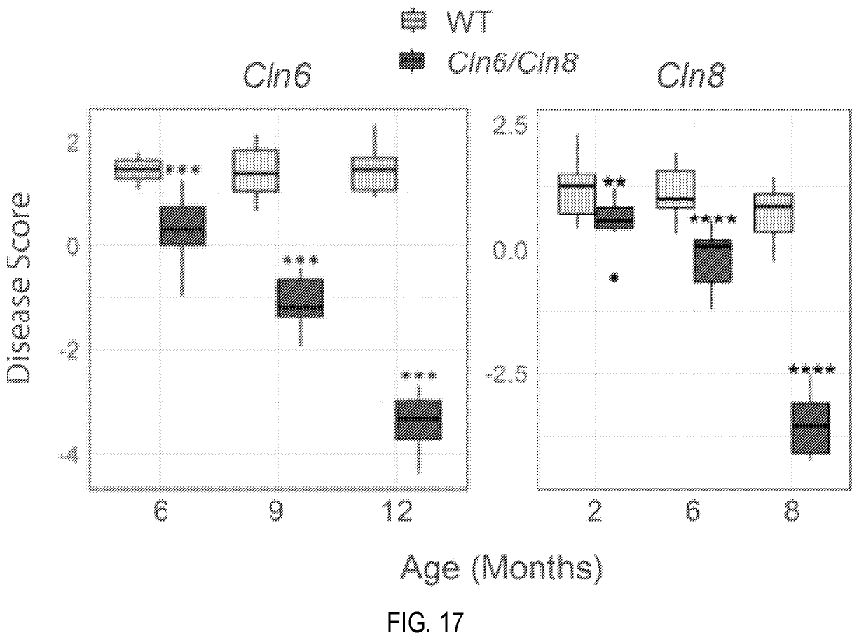

Potential biofluid biomarkers were identified based on cPCA analysis of Cln6^{nclf} and Cln8^{mnd} mice relative to a healthy mice. For CLN6 mice, the cPCA analysis comprised of MRI whole brain volume, KGA, FDG-PET (forebrain) and DTI (forebrain). Similarly, for CLN8 patients, the PCA analysis comprises of MRS, KGA, MRI cortical volume, DTI (forebrain). The results of the PCA analysis for CLN6 and CLN8 patients are summarized in FIG. 17.

### Example 3: Identifying Potential Biofluid Biomarker for CLN1 Disease

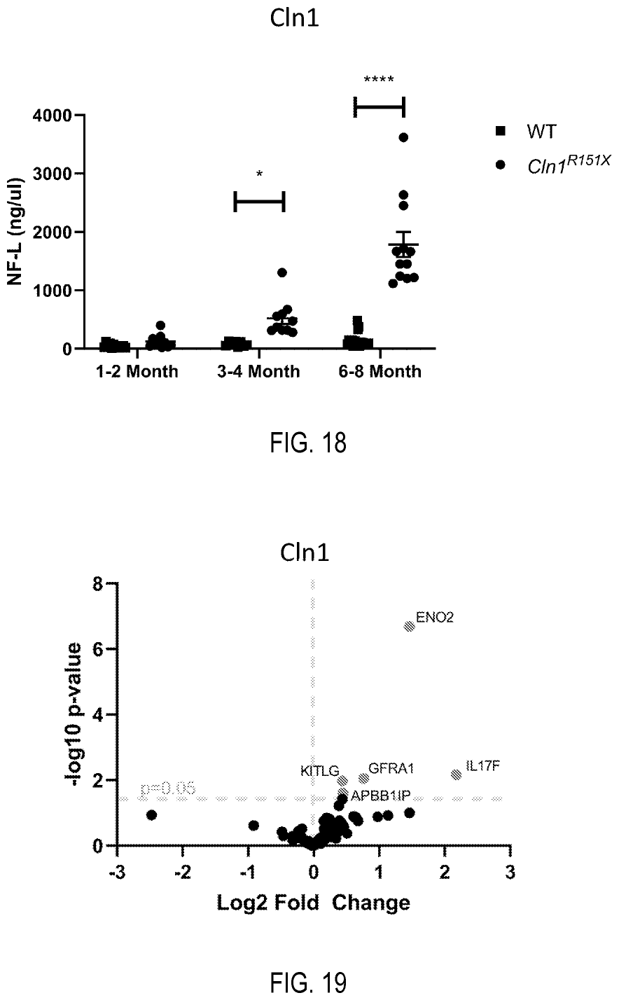

Model animals for CLN1 disease were studied. A proteomic profiling has revealed several candidate biomarkers the disease. FIG. 18 shows our analysis revealing longitudinal increase in NFL in Cln1^{R151X} mice. NFL was measured on WT and CLN1 animal serum samples from four time points using the Quanterix SIMOA platform. Significant (p<0.05) elevations were observed at 6, 24-27, 36 and 47-50 months of age. Two-way ANOVA with Tukey's post-hoc test was performed to determine degree of elevation.

In additional to NFL, our proteomic profiling has revealed several candidate biomarkers with far greater separation between WT and CLN1 animals. In FIG. 18, each target was plotted with log₂ Fold Change on the x-axis and −log₁₀ p-value (genotype difference) on the y-axis. Relative potential for each target as a biomarker was correlated with distance from the origin on the graph. Highest potential candidates were denoted by grey round points on the graph. Targets above the horizontal dashed grey line are significantly (p<0.05) different between genotypes. The vertical dashed grey line signifies a fold change of “0”. An analysis of FIG. 19 indicates a small subset of protein biomarkers, KITLG, GFRA1, APBB1IP, IL17F and ENO2, that showed substantially changed in animal model for CLN1 disease.

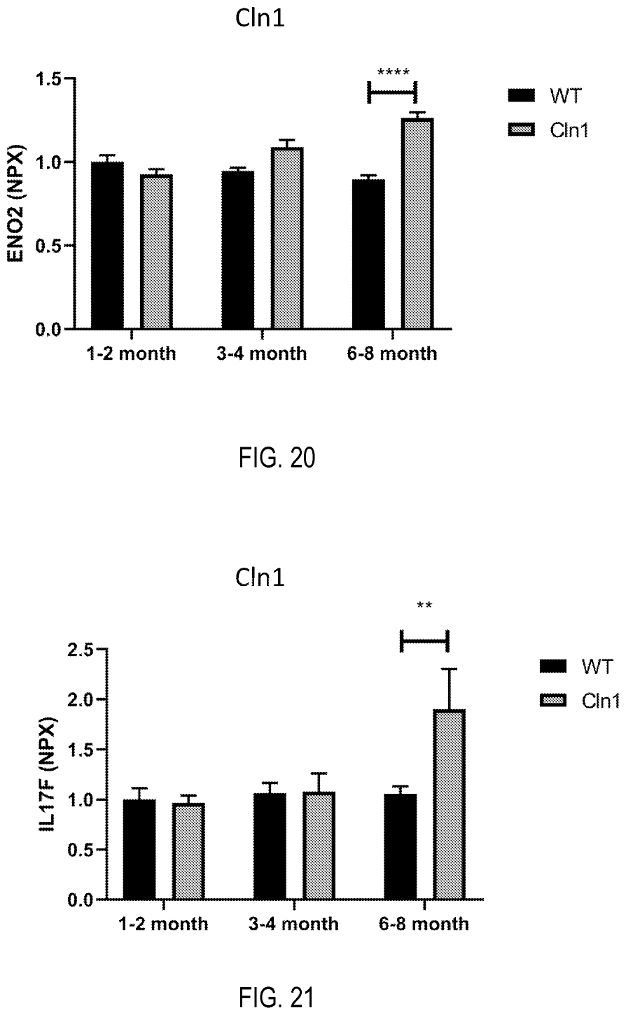

FIG. 20 shows relative levels of ENO2 in WT and CLN1 animals at 1-2, 3-4, and 6-8 months of age. Similarly, FIG. 21 shows relative levels of IL17F in WT and CLN1 animals at 1-2, 3-4, and 6-8 months of age.

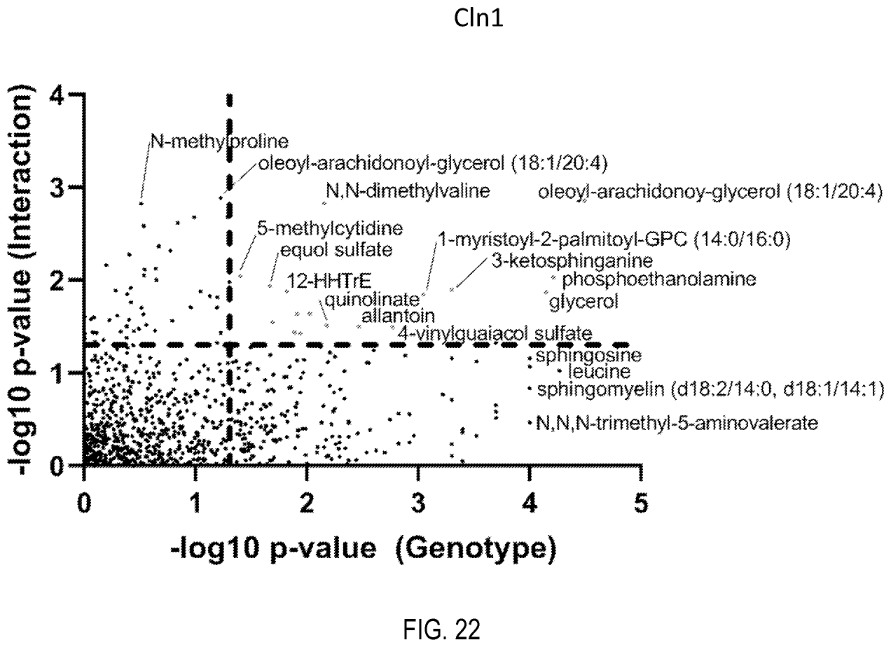

In FIG. 22, each target was plotted with log₁₀ on the x-axis and −log₁₀ p-value (genotype difference) on the y-axis. Relative potential for each target as a biomarker was correlated with distance from the origin on the graph. Highest potential candidates were denoted by grey round points on the graph. An analysis of FIG. 22 shows oleoyl-arachidonoyl-glycerol (18:1/20:4), oleoyl-arachidonoy-glycerol (18:1/20:4), N,N-dimethylvaline, 5-methylcytidine, equol sulfate, 12-HHTrE, quinolinate, allantoid, 4-vinylguaiacol sulfate, glycerol, phosphoethanolamine, 3-ketospinganine, and 1-myristoyl-2palmitoyl-GPC (14:0/16:0) have the highest potential as a biomarker for CLN 1.

### Example 4: Identifying Potential Biofluid Biomarker for CLN6 Disease

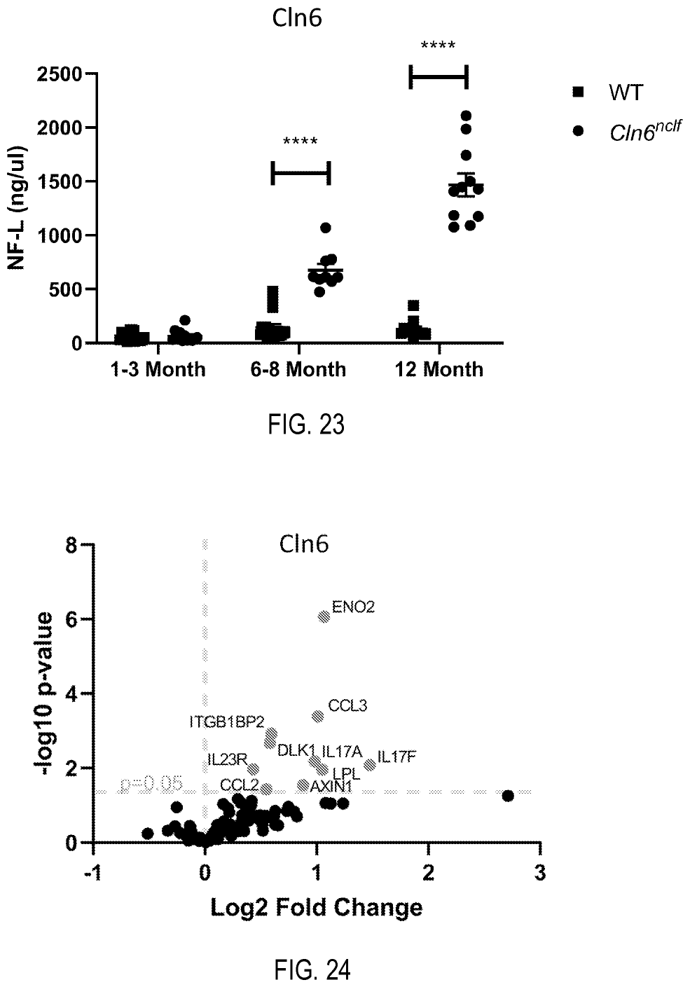

Model mice for CLN6 disease (Cln6^{nclf}) were studied. A proteomic profiling has revealed several candidate biomarkers the disease. FIG. 23 shows our analysis revealing longitudinal increase in NFL in CLN6 animals. NFL was measured on WT and CLN6 animal serum samples from four time points using the Quanterix SIMOA platform. Significant (p<0.05) elevations were observed at 6, 24-27, 36 and 47-50 months of age. Two-way ANOVA with Tukey's post-hoc test was performed to determine degree of elevation.

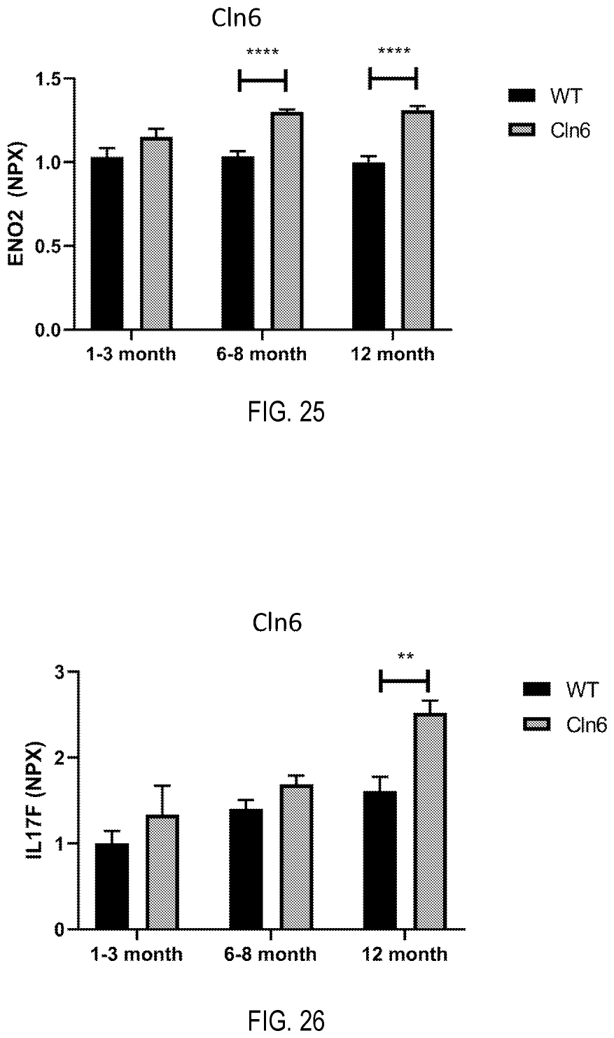

In additional to NFL, our proteomic profiling has revealed several candidate biomarkers with far greater separation between WT and CLN6 animals. In FIG. 24, each target was plotted with log₂ Fold Change on the x-axis and −log₁₀ p-value (genotype difference) on the y-axis. Relative potential for each target as a biomarker was correlated with distance from the origin on the graph. Highest potential candidates were denoted by grey round points on the graph. Targets above the horizontal dashed grey line are significantly (p<0.05) different between genotypes. The vertical dashed grey line signifies a fold change of “0”. An analysis of FIG. 25 indicates a small subset of protein biomarkers, ITGB1BP2, IL23R, CCL2, DLK1, IL17A, LPL, AXIN1, IL17F, CCL3, and ENO2, that showed substantially changed in animal model for CLN6 disease.

FIG. 25 shows relative levels of ENO2 in WT and CLN6 animals at 1-2, 3-4, and 6-8 months of age. Similarly, FIG. 26 shows relative levels of IL17F in WT and CLN6 animals at 1-2, 3-4, and 6-8 months of age.

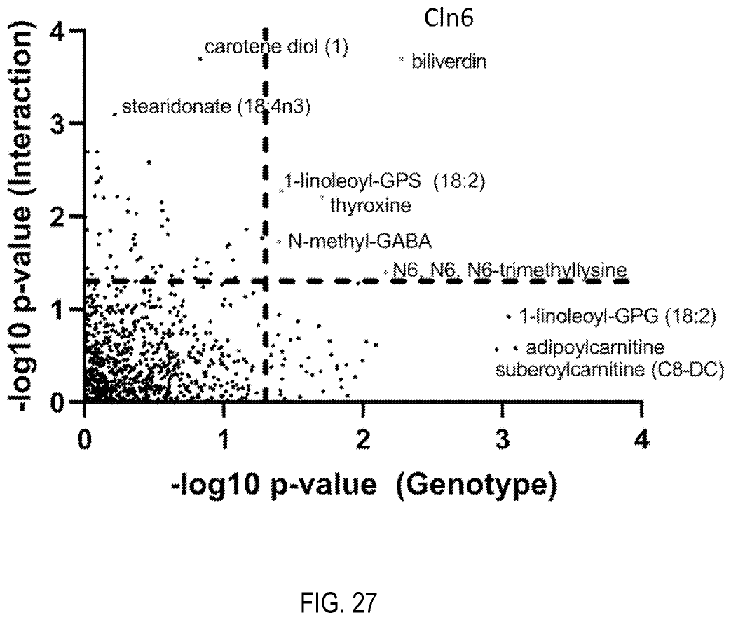

In FIG. 27, each target was plotted with log₁₀ on the x-axis and −log₁₀ p-value (genotype difference) on the y-axis. Relative potential for each target as a biomarker was correlated with distance from the origin on the graph. Highest potential candidates were denoted by grey round points on the graph. An analysis of FIG. 27 shows biliverdin, 1-linoleoyl-GPS (18:2), thyroxine, N-methyl-GABA, and N6, N6, N6-trimethyllysine have the highest potential as a biomarker for CLN 6.

### Example 5: Validation of Biomarker for CLN3 Disease in Model Animal

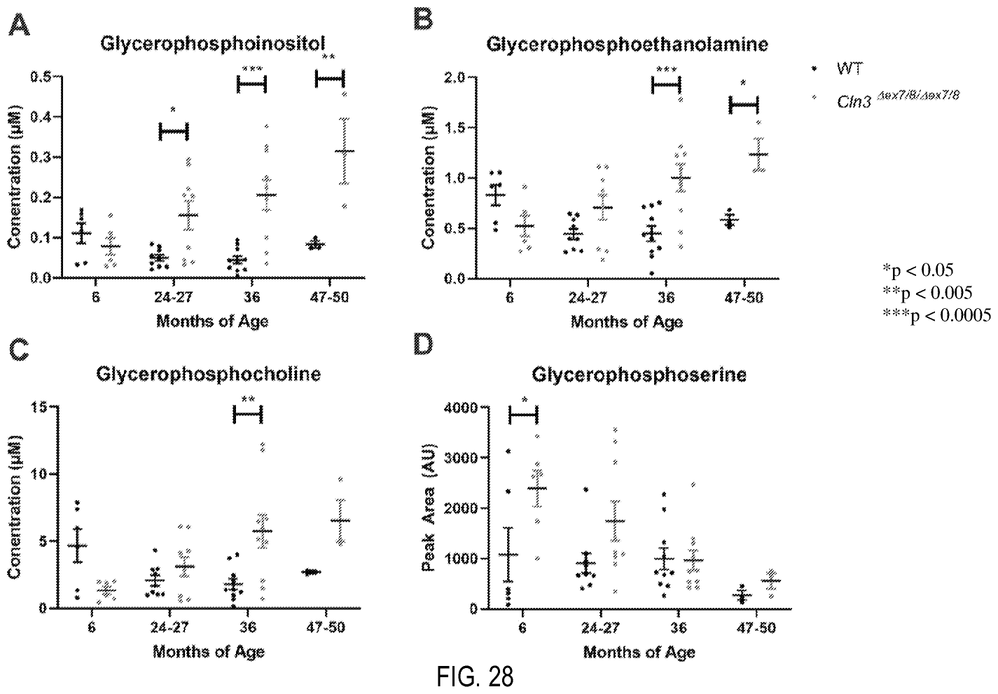

FIG. 28 shows an analysis of serum concentration of glycerophosphoinositol, glycerophosphoethanolamine (GPE), glycerophosphoserine, and glycerophosphorylcholine (GPC) in Cln3^{Δex7/8} pigs using an orthogonal semi-quantitative UHPLC-MS/MS platform. Commercial standards were used for GPI, GPE, and GPC enabling fully quantitative evaluation. For analysis, two-way ANOVA with two-tailed Sidak's multiple comparisons test was used. The analysis shows serum elevations of glycerophosphoinositol, glycerophosphoethanolamine (GPE), glycerophosphoserine, and glycerophosphorylcholine (GPC) in Cln^{3Δex7/8} pigs.

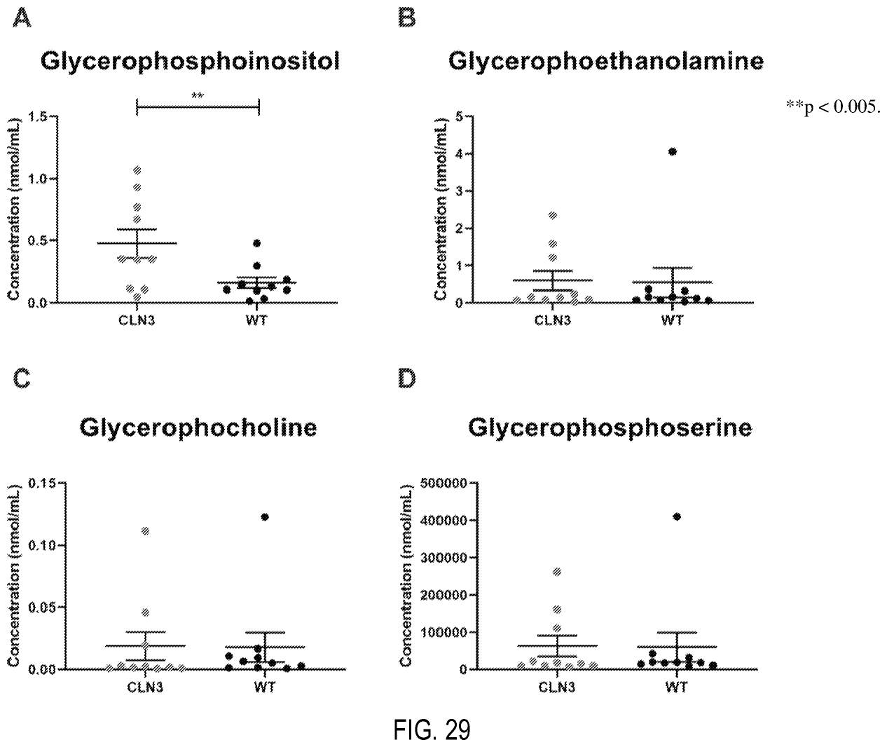

FIG. 29 shows an analysis of cerebrospinal fluid (CSF) samples from 36-month-old CLN3^{Δex7-8} and wild type pigs using the same platform that was used for serum concentration analysis. The analysis was performed using one-tailed unpaired t-test. The analysis found significant elevation of GPI in cerebrospinal fluid (CSF) but not GPE, GPC, or GPS.

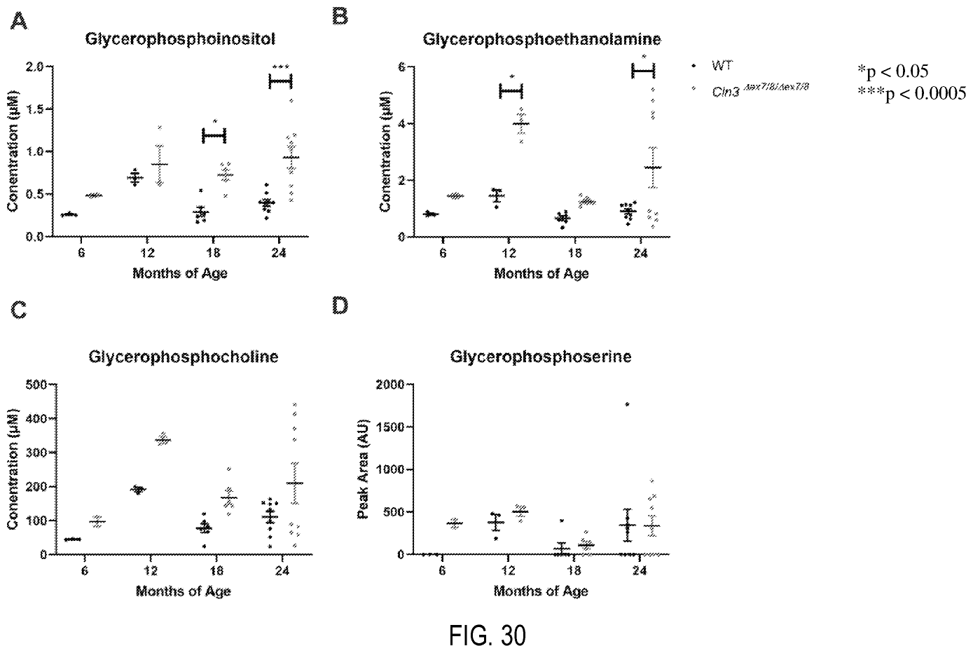

FIG. 30 shows an analysis of longitudinal plots of glycerophosphodiester levels in wild type and Cln3^{Δex7-8} mice. The analysis shows quantification of glycerophosphoinositol, glycerophosphoethanolamine (GPE), glycerophosphoserine, and glycerophosphorylcholine (GPC) in serum samples from male and female homozygous Cln3^{Δex7-8} and wild type mice at six, 12, 18, and 24 months of age. The analysis was performed using two-way ANOVA with two-tailed Sidak's multiple comparisons test. The analysis shows that GPI and GPE were significantly elevated at multiple time points. However, GPC and GPS did not show statistically significant increment in concentration.

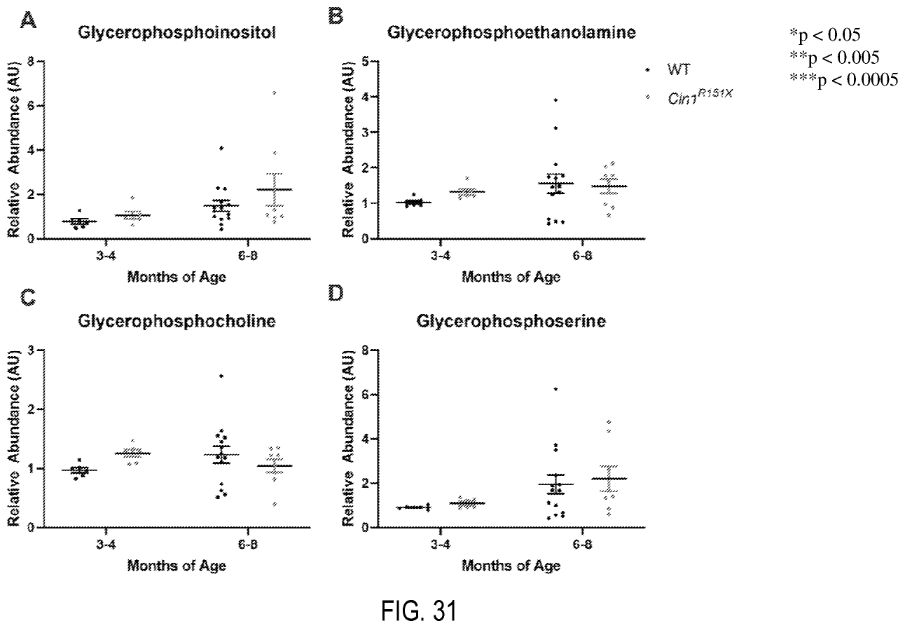

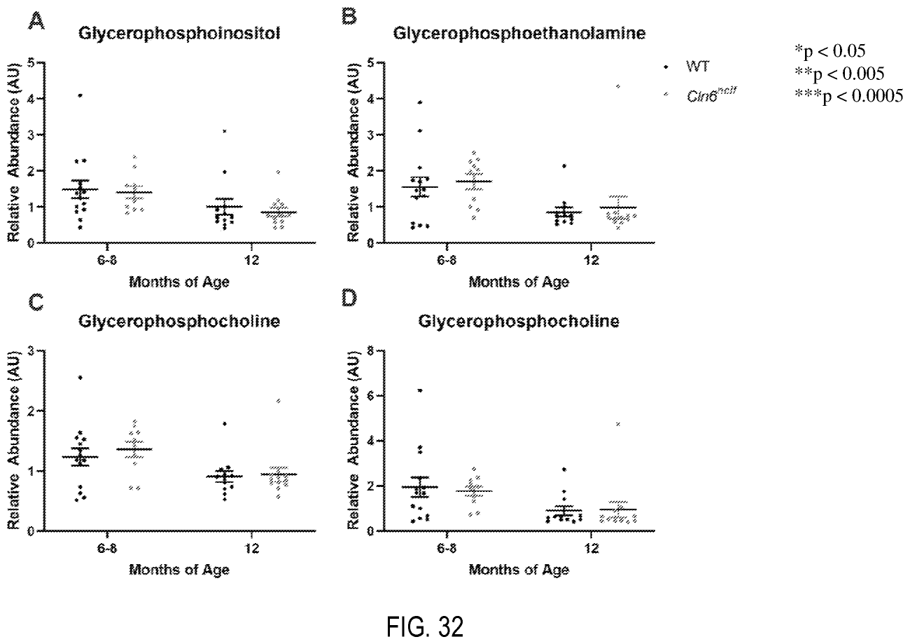

Serum samples from mouse models for two other genetic forms of Batten disease were examined to further examine whether these changes were specific to CLN3 disease. FIG. 31 shows an analysis of glycerophosphodiester levels in wild type and Cln1^{R151X} mice. FIG. 32 shows an analysis of glycerophosphodiester levels in wild type and Cln6^{nclf} mice. The analysis was performed using two-way ANOVA with two-tailed Sidak's multiple comparisons test. As shown in FIG. 31 and FIG. 32, none of the glycerophosphodiesters were elevated in serum samples from mouse models of CLN1 or CLN6 Batten disease.

Collectively, the patterns observed across species and time points suggested that the glycerophosphodiesters species may be closely linked to CLN3 disease etiology and could thus be useful as clinical biomarkers of disease status.

### Example 6: Validation of Biomarker for CLN3 Disease in Human

As shown above, Cln3^{Δex7/8} pigs exhibited significant serum elevation of glycerophosphoinositol, glycerophosphoethanolamine (GPE), glycerophosphoserine, and glycerophosphorylcholine (GPC). Accordingly, a similar study was also performed for humans diagnosed with CLN3 disease to validate the biomarkers.

Levels of each of glycerophosphoinositol, glycerophosphoethanolamine (GPE), glycerophosphoserine, and glycerophosphorylcholine (GPC) were further investigated in plasma samples from 22 phenotypic individuals with CLN3 disease, 15 heterozygous carriers, and six non-affected non-carriers (NANC). The individuals with CLN3 covered a wide range of genotypes, ages, and phenotypes in Table 2.

TABLE 2
Study design for analysis of glycerophosphodiesters
levels in subjects having CLN3.

| | Age (years) | | Sex (n) | | | |
|---|---|---|---|---|---|---|
| Group (n) | Mean | Range | Male (%) | | Female (%) | |
| NANC (6) | 21.12 | 6.3-57.1 | 3 | (50) | 3 | (50) |
| Carrier (15) | 23.35 | 7.6-54.9 | 7 | (46.7) | 8 | (53.3) |
| CLN3 (22) | 22.52 | 1.6-29.1 | 11 | (50) | 11 | (50) |

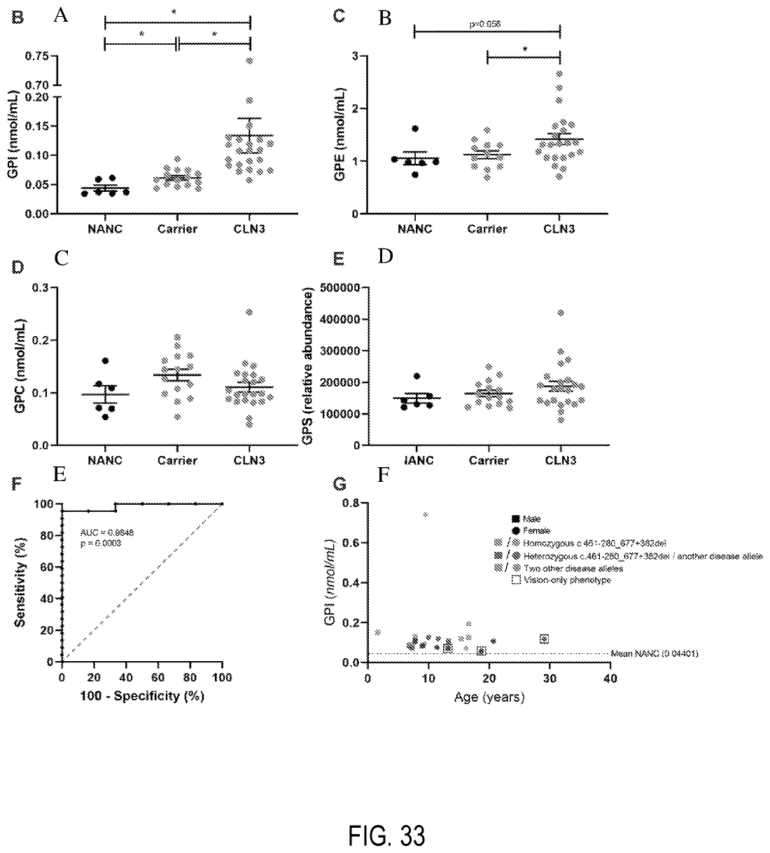

FIG. 33 shows levels of glycerophosphoinositol (upper left), glycerophosphoethanolamine (upper right), glycerophosphorylcholine (lower left), and glycerophosphoserine (lower right) in human patients having CLN3 disease.

As shown in FIG. 33, while GPS and GPC remain unchanged across the three groups, GPE and GPI were significantly elevated in the CLN3 samples, with GPI exhibiting the clearest separation. GPI demonstrated excellent sensitivity and specificity as a biomarker, with a receiver operating characteristic (ROC) area under the curve (AUC) of 0.9848 (p=0.0003, FIG. 33F) when comparing CLN3 and NANC.

CLN3 affected individuals exhibited a wide range of plasma GPI levels ranging from 0.05749 to 0.7417 nmol/mL with a mean of 0.1338 nmol/mL. The entirety of this range is substantially elevated over the mean for NANC (0.04401 nmol/mL). However, a correlation between GPI levels in CLN3 affected individuals and other demographic and phenotypic data points was inconclusive.

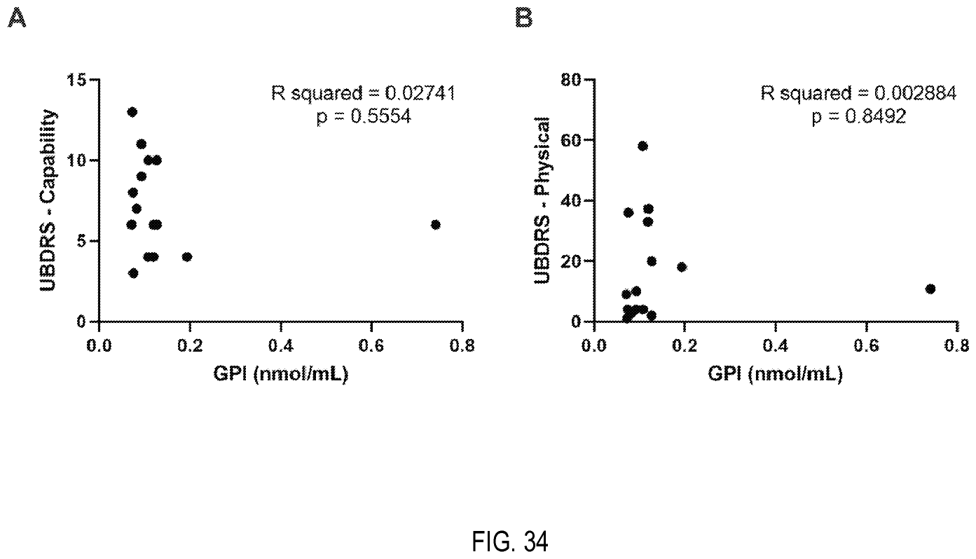

Additionally, as shown in FIG. 34, GPI levels in the CLN3 group were not significantly impacted by sex, age, or genotype, and did not correlate significantly with clinical severity as measured by United Batten Disease Rating Scale (UBDRS) scores.

Surprisingly, GPI was also elevated to intermediate levels in heterozygous carriers with a mean of 0.06150 nmol/mL. These levels are statistically different from both individuals with CLN3 and NANC. Since CLN3 carriers are free from any observable CLN3 disease process and would thus not be expected to exhibit changes in markers related to the neuroinflammation, neurodegeneration, and neuronal dysfunction, these intermediate levels strongly suggest that GPI could be closely linked to upstream CLN3 disease etiology or even to the function of CLN3. Alternatively, CLN3 could have a direct influence on metabolic pathways that generate, consume, or transport glycerophosphodiesters.

As demonstrated, GPE and GPI could have utility as biomarkers of CLN3 disease status. GPI, in particular, shows consistent elevations across a diverse cohort of individuals with CLN3. This raises the potential to use these biomarkers as a blood-based diagnostic test (e.g., a newborn screen) or as an efficacy measure for disease-modifying therapies. Therapies that restore CLN3 function may reduce GPI in various tissues and biofluids. CSF levels, in particular, may be valuable for monitoring the efficacy of CNS-directed therapies. Additionally, combining GPI or GPE with biomarkers that correlate with clinical severity may provide for a comprehensive assessment of molecular and clinical disease status.

1. A method of determining a disease score of a patient diagnosed with lysosomal storage disease (LSD), wherein the disease score comprises one or more of the following: (i) one or more biofluid biomarkers; (ii) one or more neurophysiological measurements; and (iii) one or more neurobehavior measurement.

2. The method of claim 1, wherein the one or more biomarkers is selected from the group consisting of one or more metabolites, one or more proteins, one or more lipids, and one or more lipid conjugates.

3. The method of claim 2, wherein the one or more metabolites is selected from the group consisting of glycerophosphoinositol (GPI), glycerophosphocholine (GPC), glycerophosphoserine (GPS), and glycerophosphoethanolamine (GPE).

4. The method of claim 2, wherein the one or more proteins is selected from the group consisting of neurofilament light (NFL), ubiquitin c-terminal hydrolase L1 (UCHL1), mitochondrial ATP synthase subunit C (SCMAS), gamma enolase (ENO2), cathepsin D (CTSD), Progranulin (GRN), palmitoyl-protein thioesterase 1 (PPT1), tripeptidyl-peptidase 1 (TPP1), troponin T, and troponin I.

5. The method of claim 2, wherein the one or more lipids is selected from the group consisting of Phosphatidylcholine, Phosphatidylcholine, Phosphatidylcholine and Phosphatidylethanolamine.

6. The method of claim 2, wherein the one or more lipid conjugates is selected from the group consisting of 1-stearoyl-2-docosahexaenoyl-GPC (18:0/22:6), 1-palmitoyl-2-docosahexaenoyl-GPE (16:0/22:6), 1-stearoyl-2-docosahexaenoyl-GPC (18:0/22:6), and 1-oleoyl-2-docosahexaenoyl-GPC (18:1/22:6).

7. The method of claim 1, wherein the one or more neurophysiological measurements is selected from the group consisting of gait analysis, neuro-imagining metrics, retinal function, peripheral nerve function, clinical assessment of neurological function, and patient-reported episodes.

8. The method of claim 7, wherein the neuro-imagining metrics include one or more of: brain ventricle size, grey matter hyperintensities or hypointensities, white matter hyperintensities or hypointensities, periventricular hyperintensities, cerebellar atrophy, cortical atrophy, whole brain volume, corpus callosum volume, white matter integrity, radial diffusivity, axial diffusivity, and magnetic resonance spectroscopy measurements.

9. The method of claim 7, wherein the retinal function includes one or more of: ERG waveform, visual-evoked potential measurement, and optical coherence tomography measurement.

10. The method of claim 1, wherein the one or more neurobehavioral measurements include measuring one or more of: motor function, language function, cognitive function, clinical rating scale, and PROM.

11. The method of claim 1, wherein the LSD is CLN1, CLN2, CLN3, CLN4, CLN5, CLN6, CLN7, CLN8, CLN10, CLN11, CLN12, CLN13, and/or CLN14; Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy, Farber lipogranulomatosis, Wolman and cholesteryl ester storage disease, pycnodystostosis, cystinosis, Salla disease, Danon disease, Griscelli disease Types 1/2/3, and Hermansky Pudliak Disease Type 2.

12. The method of claim 1, wherein the LSD is CLN1, CLN2, CLN3, CLN4, CLN5, CLN6, CLN7, CLN8, CLN10, CLN11, CLN12, CLN13, and/or CLN14.

13. A method of treating lysosomal storage disease (LSD) patient, the method comprising:
determining the disease score according to the method of claim 1; and
administering the patient a therapy, wherein the therapy comprises one or more of the following: (i) enzyme replacement therapy, (ii) gene therapy; and (iii) a small molecule.

14. The method of claim 13, wherein the therapy is a gene therapy.

15. The method of claims 13, wherein the gene therapy is delivered systemically or to central nervous system.

16. The method of claim 15, wherein the gene therapy is delivered to a brain.

17. The method of claim 15, wherein the therapy is delivered to a spinal cord.

18. The method of claim 14, wherein the gene therapy is delivered intrathecally.

19. The method of claim 14, wherein the gene therapy includes a composition comprising rAAV9.

20. The method of claim 19, wherein the rAAV9 comprises a self-complementary genome comprising said polynucleotide.
